(19) United States
(12) Patent Application Publication
(10) Pub. No.: US 2026/0086637 A1
(43) Pub. Date: Mar. 26, 2026

(54) RING-SHAPED WEARABLE DEVICES, SYSTEMS, AND METHODS OF USE THEREOF

(71) Applicant: Lotus Laboratories Inc., San Jose, CA (US)

(72) Inventors: Dhaval PATEL, San Jose, CA (US); Andrew LE, Austin, TX (US)

(73) Assignee: Lotus Laboratories Inc., San Jose, CA (US)

(21) Appl. No.: 18/877,117

(22) PCT Filed: Aug. 2, 2023
(86) PCT No.: PCT/US2023/071551

§ 371 Date: Dec. 19, 2024

Related U.S. Application Data
(60) Provisional application No. 63/394,515, filed on Aug. 2, 2022.

(51) Int. Cl. G06F 3/01; A61B 5/00; F21V 23/04; H01H 3/26; H01H 3/28; H02J 50/00; H02J 50/90; H02J 105/44; H05B 47/175; H05B 47/19; H05B 47/195

(52) U.S. Cl. CPC G06F 3/014; F21V 23/0435; H01H 3/26; H01H 3/28; H02J 50/001; H02J 50/90; H05B 47/19; H05B 47/195; H05B 47/1965; A61B 5/0022; A61B 5/6802; H01H 2003/266; H02J 2105/44

(57) ABSTRACT

The disclosure provides wearable devices, comprising a ring-shaped body, input devices, and output devices, designed for users, including users with one or more disabilities. The disclosure also provides systems for wireless control of receiver devices, comprising wearable devices, removable power storage systems, charging stations, and receiver devices or smart receiver devices. Wearable devices of the present disclosure are configured to be modular and interchangeable, and systems of the present disclosure are extensible to the internet and smart devices without requiring rewiring of preexisting devices. Also provided herein are methods of using such wearable devices, receiver devices, smart receiver devices, and systems.

### CROSS-REFERENCE TO RELATED APPLICATION

The present application hereby claims the benefit of priority to U.S. application Ser. No. 63/394,515, filed Aug. 2, 2022, which is incorporated by reference herein in its entirety.

### TECHNICAL FIELD

The present technology relates generally to devices, systems, and methods related to wearable devices. In some embodiments, the present technology is directed to devices, systems, and methods for remotely managing systems of an indoor environment by persons with physical limitations.

### BACKGROUND OF THE INVENTION

Portable electronic devices are now commonplace throughout much of the world and serve a variety of functions, including controlling other electronic devices. Smartphones, tablet computers, eBook readers, and laptop computers are sufficiently small and lightweight to be carried, and include onboard power storage devices and wireless communications units so they may operate without wired connections. However, these portable devices are not always within a user's reach, their operation generally requires the use of two hands and/or other means of input and that a user look at the device, and their operation can be time-consuming and difficult to navigate for users lacking tech savvy. In particular, devices with touch screens require both use of two hands and that a user look at the device; users not able to perform both activities are not able to use the device. Wearable electronic devices address only one of these difficulties—devices not always within a user's reach or carried on person.

Wearable devices, such as glasses, watches, armbands, hearing aids, and rings, may be worn on a part of the user's body instead of physically held in a user's hands. These devices exhibit a variety of form factors, including various geometries and sizes with reduced footprints. Wearable device form factors impact comfort, appearance, and ease of use.

Operation of these wearable devices generally requires the use of two hands and often requires the user to interact with a graphical user interface (GUI) on a screen or provide voice commands as input. This presents a challenge for some users who either permanently or temporarily lack the use of one or both hands, eyes, or voice. In particular, users lacking fine motor control may find the small wearable device touchscreens very challenging. Users lacking gross-motor control, for example due to stroke or quadriplegia, may find it difficult to use such wearable devices because they can neither lift their arm to view the small wearable device touchscreen nor touch it with both hands. Users lacking sight may find navigating a GUI on a small wearable device touchscreen using a screen-reader or a screen magnifier very slow and/or cumbersome. Furthermore, wearable devices generally must be removed to recharge them, rendering them useless while they are being recharged

In the United States, the ADA Standards for Accessible Design, published on Sep. 15, 2010, and incorporated by reference herein, provides guidance on public building regulations for persons with disabilities under the Americans with Disabilities Act of 1990. Under 28 CFR 35.151, public facilities must be “readily accessible to and usable by individuals with disabilities.” The Fair Housing Design Manual, incorporated by reference herein, provides guidance on regulations concerning accessible design for new construction of multifamily dwellings. These regulations include requirements for accessibility of environmental controls such as light switches and thermostats. The U.S. Green Building Council, issuer of the LEED standards, promotes “inclusive design” that includes assistive technology “controls for devices and systems affecting occupancy of the space and user comfort, including but not limited to lighting, window shades, and thermostats.” Various countries around the world encouraging aging in place through regulations and incentives related to adaptive control of the internal environment.

Thus, there is a persistent need for wearable devices adapted for comfort, appearance, and ease of use for users of all abilities and disabilities, as well as systems and methods of using such wearable devices for controlling other electronic devices thereby providing control of various internal environments and making them accessible and inclusive.

### BRIEF SUMMARY OF THE INVENTION

Thus, in accordance with the present disclosure, wearable devices adapted for comfort, appearance, and ease of use are provided herein. The disclosure features wearable devices with a ring-shaped body, input devices, and output devices. The disclosure also features systems for wireless control of receiver devices to reconfigure a position, and methods of using such wearable devices, receiver devices, and systems to reconfigure a position. The disclosure also features systems for wireless control of smart receiver devices to cause smart devices to perform functions, and methods of using such wearable devices, smart receiver devices, and systems to perform functions.

The disclosure provides a wearable device comprising a ring-shaped body, one or more input devices provided on at least one region of an inner circumferential surface or on at least one region of an outer circumferential surface, and one or more output devices provided on at least one region of the inner circumferential surface or at least one region of the outer circumferential surface.

In some embodiments, the one or more input devices is selected from the group consisting of a touch sensor, a sound sensor, a motion sensor, and a button. In some embodiments, the one or more output devices is selected from the group consisting of a haptic source, a sound source, and an electromagnetic radiation source. In some embodiments, the haptic source is configured to generate haptic signals. In some embodiments, the sound source comprises a speaker assembly configured to generate sound signals. In some embodiments, the electromagnetic radiation source is configured to generate one or more electromagnetic radiation signals. In some embodiments, the electromagnetic radiation source comprises a light emitting diode. In some embodiments, the one or more electromagnetic radiation signals comprises one or more visible light signals or one or more invisible light signals. In some embodiments, the one or more invisible light signals comprises infrared radiation or radio frequency radiation.

In some embodiments, the wearable device further comprises one or more antenna assemblies, wherein the one or more antenna assemblies is configured on the outer circumferential surface. In some embodiments, the wearable device further comprises one or more wireless communication units. In some embodiments, the wearable device further comprises one or more processors. In some embodiments, the one or more processors is configured to control one or more functions of the wearable device. In some embodiments, the wearable device further comprises memory storing instructions executable by the one or more processors. In some embodiments, the memory stores instructions executable by the one or more processors, which when executed cause the wearable device to detect sounds, recognize speech commands, and communicate with one or more voice assistants.

In some embodiments, the one or more wireless communication units is configured for wireless communication over a Bluetooth protocol, over a Near-Field Communication protocol, RFID protocol, over Wi-Fi, over a mesh network, over ultra-wideband, over radio frequency, over infrared, over cellular communications, or over the Global Positioning System. In some embodiments, the one or more wireless communication units is configured for wireless communication over a Bluetooth protocol or a mesh protocol with one or more hubs. In some embodiments, the one or more hubs is configured to interface with the internet. In some embodiments, the one or more hubs is selected from the group consisting of the charging station, a mobile device, and a smart device.

In some embodiments, the wearable device further comprises one or more power storage devices configured to store electric power. In some embodiments, the one or more power storage devices is removable and interchangeable. In some embodiments, the one or more power storage devices is configured to charge at a charging station. In some embodiments, the charging station is configured to transmit electric power to the one or more power storage devices. In some embodiments, the one or more power storage devices is configured for inductive or non-inductive wireless charging by pressure contacts. In some embodiments, magnets are configured to align components for pressure contact. In some embodiments, the wearable device further comprises one or more energy harvesting devices, wherein the one or more energy harvesting devices is configured to transmit electric power to the one or more power storage devices, and wherein the one or more energy harvesting devices is a thermoelectric generator or a transducer such as a piezo.

In some embodiments, the wearable device further comprises one or more biometric sensors configured to sense and collect biometric information. In some embodiments, the one or more biometric sensors is configured on the inner circumferential surface. In some embodiments, the one or more biometric sensors is selected from the group consisting of a heart rate sensor, an oxygen saturation sensor, a temperature sensor, a blood pressure sensor, and a glucose sensor. In some embodiments, the one or more biometric sensors comprises a first temperature sensor on at least one region of the inner circumferential surface and configured to sense a user's temperature, and a second temperature sensor on at least one region of the outer circumferential surface and configured to sense an ambient temperature.

In some embodiments, the touch sensor comprises a capacitive sensor, an inductive sensor, an optical sensor, or a combination thereof. In some embodiments, the pressure sensor is configured on the outer circumferential surface. In some embodiments, the fingerprint sensor is configured on at least one region of the inner circumferential surface.

In some embodiments, the sound sensor comprises one or more microphones. In some embodiments, the one or more microphones comprises a first microphone configured to detect sounds and a second microphone to detect sounds, wherein the first microphone and the second microphone are configured for active noise cancellation.

In some embodiments, the wearable device further comprises an accelerometer, a gyroscope, and a magnetometer. In some embodiments, the motion sensor is configured to receive information from the accelerometer, the gyroscope, and the magnetometer. In some embodiments, the motion sensor and the one or more processors are configured to calculate displacement, velocity, acceleration, and rotational motion.

In some embodiments, the wearable device further comprises a tactile surface configured to orient the wearable device with respect to a user's finger. In some embodiments, the wearable device further comprises a power storage device indicator light configured on the outer circumferential surface.

In some embodiments, the wearable device is embodied as a ring comprising a first ring and a second ring, wherein the first ring and the second ring are configured to share a center. In some embodiments, the first ring is removable from the second ring. In some embodiments, the first ring is proximal to the center and the second ring is distal to the center. In some embodiments, the first ring is nested within the second ring, and in some embodiments the first ring is adjacent to the second ring. In some embodiments, the one or more biometric sensors is configured on the inner circumferential surface of the first ring nested within the second ring. In some embodiments, the wearable device is embodied as a ring configured to be placed on and surround a user's finger.

The disclosure provides a system comprising one or more wearable devices and one or more receiver devices configured to communicate with the one or more wearable devices. In some embodiments, the one or more receiver devices comprises a receiver wireless communication unit, one or more receiver processors, one or more receiver power storage devices, an electromagnet, one or more position sensors, and receiver memory. In some embodiments, the receiver memory stores receiver instructions executable by the one or more receiver processors, which when executed cause the one or more receiver devices to detect a position of the electromagnet, actuate the electromagnet, and reconfigure the position.

In some embodiments, the receiver wireless communication unit comprises a receiver electromagnetic radiation sensor configured to detect the one or more electromagnetic radiation signals emitted from the electromagnetic radiation source of the one or more wearable devices. In some embodiments, the electromagnet is selected from the group consisting of a solenoid, a servomotor, a stepper motor, and a motor.

In some embodiments, the one or more receiver devices further comprises a receiver electromagnetic radiation source configured to generate one or more receiver electromagnetic radiation signals, and the one or more wearable devices further comprises an electromagnetic radiation sensor configured to detect the one or more receiver electromagnetic radiation signals emitted from the receiver electromagnetic radiation source of the one or more receiver devices.

The disclosure provides a system comprising one or more wearable devices, and one or more smart receiver devices configured to communicate with the one or more wearable devices. In some embodiments, the one or more smart receiver devices comprises a smart receiver wireless communication unit, one or more smart receiver power storage devices, one or more smart receiver processors, and smart receiver memory. In some embodiments, the smart receiver wireless communication unit comprises a smart receiver electromagnetic radiation sensor configured to detect the one or more electromagnetic radiation signals emitted from the electromagnetic radiation source of the one or more wearable devices. In some embodiments, the one or more smart receiver devices further comprises a smart receiver electromagnetic radiation source configured to generate one or more smart receiver electromagnetic radiation signals, and the one or more wearable devices further comprises an electromagnetic radiation sensor configured to detect the one or more smart receiver electromagnetic radiation signals emitted from the smart receiver electromagnetic radiation source of the one or more smart receiver devices.

In some embodiments, the memory stores instructions executable by the one or more processors, which when executed cause the one or more wearable devices to detect a smart receiver identification from the smart receiver memory, interface with the internet, and cause one or more smart receiver devices to perform one or more functions. In some embodiments, the memory stores instructions executable by the one or more processors, which when executed cause the one or more wearable devices to receive a smart receiver identification transmitted wirelessly by the smart receiver wireless communication unit, interface with the internet, and cause one or more smart receiver devices to perform one or more functions.

The disclosure provides a method of controlling one or more receiver devices, comprising powering on one or more wearable devices and activating the one or more input devices. In some embodiments, the activating step comprises touching the one or more wearable devices, producing one or more sound signals, or moving the one or more wearable devices. In some embodiments, the method further comprises executing instructions, detecting the position of an electromagnet, actuating an electromagnet, and reconfiguring the position. In some embodiments, the method further comprises receiving one or more receiver electromagnetic radiation signals emitted by the receiver electromagnetic radiation source, and activating one or more output devices in response to the one or more receiver electromagnetic radiation signals by wireless communication.

The disclosure provides a method of controlling one or more smart receiver devices, comprising powering on one or more wearable devices and activating the one or more input devices. In some embodiments, the method further comprises detecting a smart receiver identification from the smart receiver memory, interfacing with the internet, and causing the one or more smart devices to perform one or more functions.

The disclosure provides a method of controlling one or more receiver devices, comprising powering on one or more wearable devices of the systems disclosed herein, and activating the one or more input devices. In some embodiments, the method further comprises receiving the one or more electromagnetic radiation signals emitted by the receiver electromagnetic radiation source. In some embodiments, the method further comprises activating the one or more output devices in response to the one or more receiver electromagnetic radiation signals.

These and other embodiments are described in more detail in the detailed description below. For avoidance of doubt, the inventive technology is not limited to but rather is illustrated by the various embodiments herein.

### BRIEF DESCRIPTION OF THE DRAWINGS

FIG. 1A is a perspective view of an exemplary wearable device.

FIG. 1B is an exploded perspective view of the wearable device of FIG. 1A.

FIG. 1C is an exploded perspective view of the wearable device of FIG. 1A.

FIG. 2A is a perspective view of another exemplary wearable device.

FIG. 2B is a perspective view of the wearable device of FIG. 2A where a power storage device has been detached from the wearable device.

FIG. 2C is a perspective view of another exemplary wearable device.

FIG. 2D is a perspective view of the wearable device of FIG. 2C where two power storage devices have been detached from the wearable device.

FIG. 2E is a perspective view of another exemplary wearable device.

FIG. 2F is a top view of the wearable device of FIG. 2E.

FIG. 3 is a block diagram of a wearable device according to various embodiments of the present disclosure.

FIG. 4A depicts an exemplary wearable device disposed on a user's finger.

FIG. 4B depicts motion of the user's finger and the wearable device shown in FIG. 4A.

FIG. 4C depicts motion of the user's hand and the wearable device shown in FIG. 4A.

FIG. 4D depicts a user's thumb activating a touch sensor, a button, or both a touch sensor and a button of the wearable device shown in FIG. 4A.

FIG. 5A depicts an exemplary system, including a wearable device, a receiver device, and wireless communication between them, installed on a light switch. The light switch must be activated using the receiver device.

FIG. 5B depicts an exemplary system, including a wearable device, a receiver device, and wireless communication between them, installed on a toggle light switch. The toggle light switch may be activated manually or by wireless communication with the receiver device.

FIG. 5C depicts the system shown in FIG. 5B, including an exploded perspective view and a block diagram of the receiver device, installed on a toggle light switch.

FIG. 5D depicts an exemplary system, including a wearable device, a receiver device, and wireless communication between them, installed on a rocker light switch. The rocker light switch may be activated manually or by wireless communication with the receiver device.

FIG. 5E depicts an exploded perspective view of an exemplary system, including a wearable device, a receiver device, and wireless communication between them, installed on a rocker light switch.

FIG. 5F depicts another exemplary system, including a wearable device, a smart switch, a smart receiver device, a block diagram of the smart receiver device, a charging station, a network, and wireless communications.

FIG. 5G depicts embodiments of systems of the present invention, including exemplary hubs and wireless communication with exemplary smart devices.

FIGS. 6A-6C are flowcharts illustrating methods of activating a wearable device and controlling one or more receiver devices without requiring the internet in accordance with embodiments of devices, systems, and methods of the present disclosure.

FIGS. 7A-7C are flowcharts illustrating methods of activating a wearable device and controlling one or more receiver devices including button touch input without requiring the internet in accordance with embodiments of devices, systems, and methods of the present disclosure.

FIGS. 8A-8E concern a ring design embodiment.

FIGS. 9A-9E concern a ring design embodiment.

FIGS. 10A-10E concern a ring design embodiment.

FIGS. 11A-11E concern a ring design embodiment.

FIGS. 12A-12E concern a ring design embodiment.

FIG. 13 is an exploded view of a ring design embodiment.

FIGS. 14-17 concern switch plate design embodiments.

FIGS. 18A-18D concern switch plate design embodiments.

FIGS. 19A-19D concern rocker plate design embodiments.

### DETAILED DESCRIPTION OF CERTAIN EMBODIMENTS

The detailed description is set forth with reference to the accompanying drawings. With reference to the drawings, similar components are provided with the same reference numerals, and different reference numerals may be used to identify similar components. The representative embodiments of the drawings and described herein are presented by way of example and not by way of limitation. Some components may not be present in various embodiments, and some embodiments may use components not illustrated in the drawings. Changes may be made in the form and details of embodiments disclosed herein resulting in equivalent embodiments that remain within the scope of the accompanying claims.

### Definitions

Unless otherwise defined, all technical terms used in the description herein and in the accompanying claims have identical meaning as understood by one of ordinary skill in the art. The terminology used herein is not intended to be limiting and is used for the purpose of describing particular embodiments in the description herein.

As used herein, the use of singular terminology to describe a component may encompass a plural number of such components depending on the context. Similarly, the use of plural terminology to describe a plural number of components may encompass a single component, depending on the context.

In the description used herein, ordinal numbers such as “first”, “second”, and the like are used to identify components and do not limit the number of components. These terms are generally used only to distinguish one component from another. As used herein, the terms “inner” and “outer” are for illustrative purposes as reference positions, and are not necessarily absolute positions.

The singular forms “a,” “an,” and “the” are intended to include the plural forms as well and are consistent with the meaning of “one or more,” “at least one,” and “one or more than one,” unless the context clearly indicates otherwise.

As used herein, terms such as “include” and “including” are intended to indicate the existence of several components, functions, or steps as disclosed in the specification, and it is understood that fewer or greater components, functions, or steps may be utilized.

### Wearable Devices

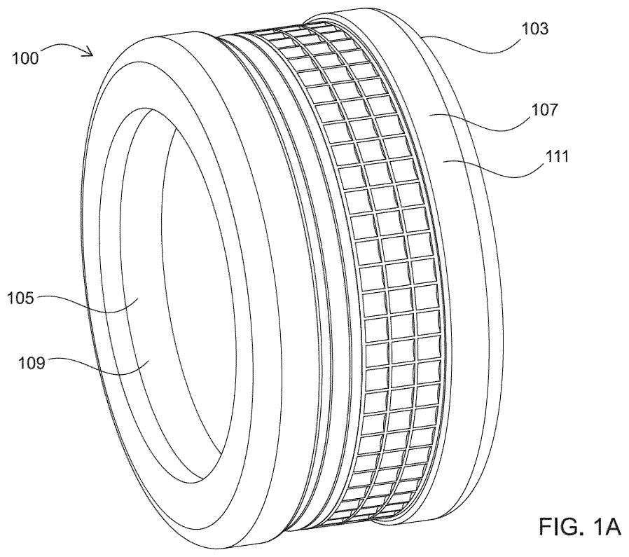

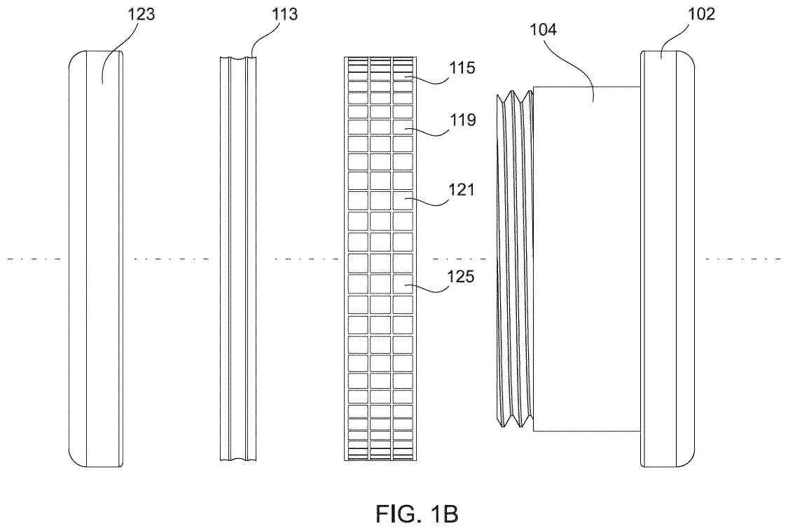

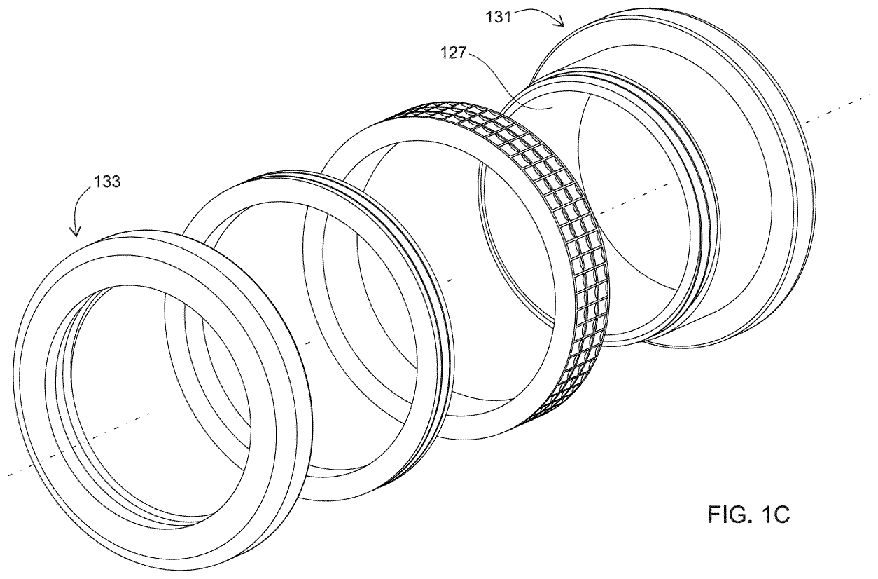

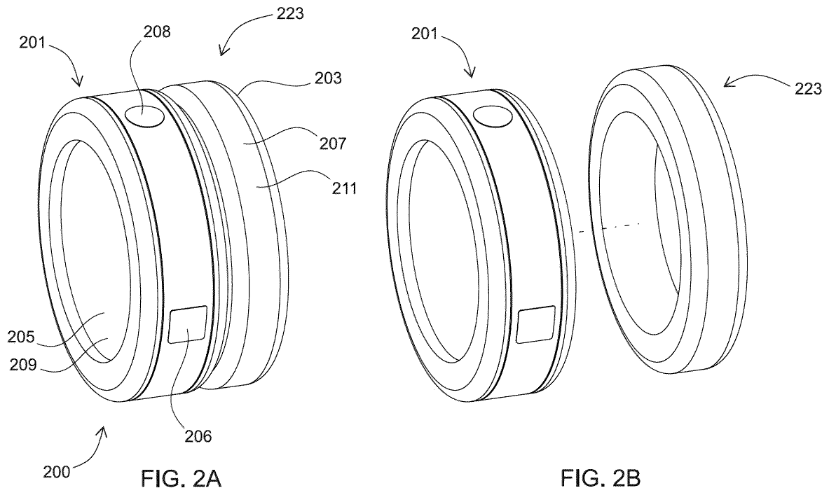

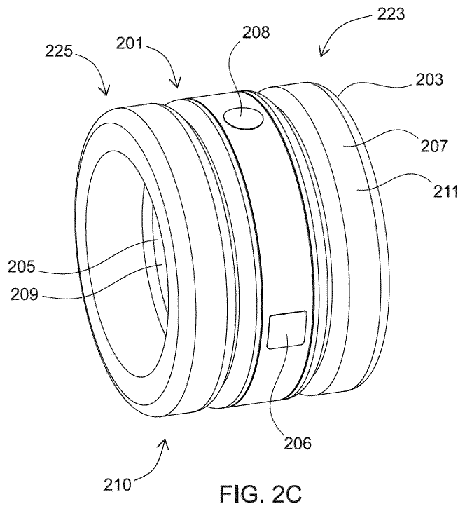

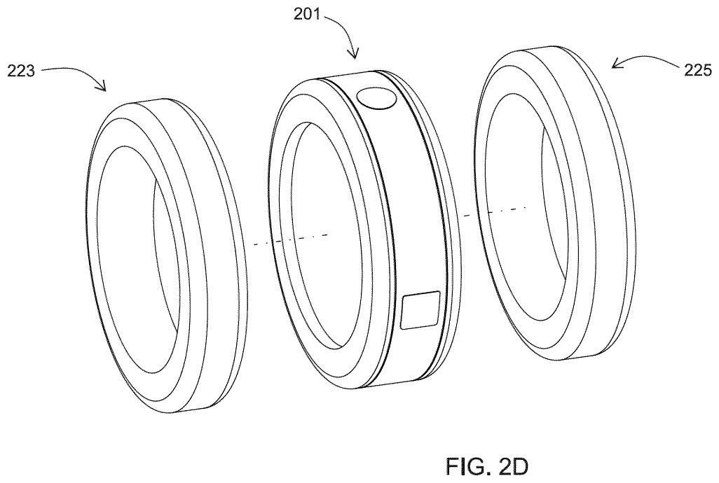

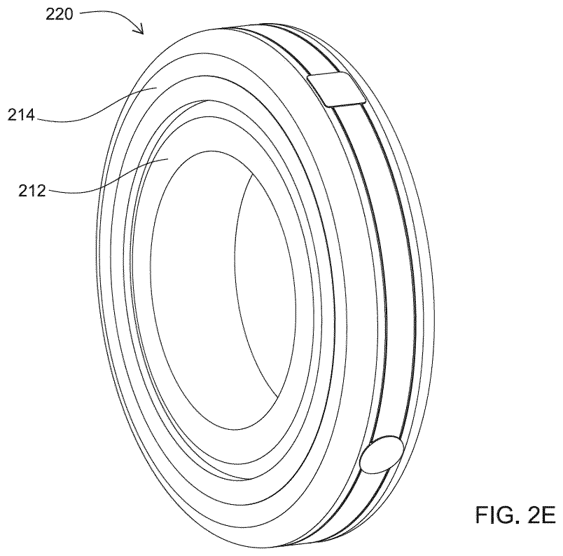

As depicted in the exemplary embodiments of FIG. 1A, FIG. 2A, FIG. 2C, and FIG. 2E, the wearable devices disclosed herein may exhibit various form factors to enhance comfort, aesthetic appearance, and ease of use.

In the exemplary embodiment of FIG. 1A, wearable device 100 is in its fully assembled state wherein a ring-shaped body 103 comprises an inner circumferential surface 105 and an outer circumferential surface 107. One or more input devices and one or more output devices may be provided on at least one region of the inner circumferential surface 109 of the annular structure of ring-shaped body 103. One or more input devices and one or more output devices may be provided on at least one region of the outer circumferential surface 111 of the annular structure of ring-shaped body 103.

In the exemplary embodiment of FIG. 2A, wearable device 200 is in its fully assembled state wherein a ring-shaped body 203 comprises an inner circumferential surface 205 and an outer circumferential surface 207. One or more input devices and one or more output devices may be provided on at least one region of the inner circumferential surface 209 of the annular structure of ring-shaped body 203. One or more input devices and one or more output devices may be provided on at least one region of the outer circumferential surface 211 of the annular structure of ring-shaped body 203. Wearable device 200 comprises a first ring 201 and a second ring 223, and first ring 201 and second ring 223 are configured to share a center.

In the exemplary embodiment of FIG. 2C, wearable device 210 is in its fully assembled state wherein a ring-shaped body 203 comprises an inner circumferential surface 205 and an outer circumferential surface 207. One or more input devices and one or more output devices may be provided on at least one region of the inner circumferential surface 209 of the annular structure of ring-shaped body 203. One or more input devices and one or more output devices may be provided on at least one region of the outer circumferential surface 211 of the annular structure of ring-shaped body 203. Wearable device 210 comprises a first ring 201, a second ring 223, and a third ring 225, and first ring 201, second ring 223, and third ring 225 are configured to share a center.

In the exemplary embodiment of FIGS. 2E and 2F, wearable device 220 comprises a first ring 212 and a second ring 214, and first ring 212 and second ring 214 are configured to share a center. In this embodiment, first ring 212 is proximal to the center and second ring 214 is distal from the center, i.e., first ring 212 is nested within second ring 214. In this embodiment, the first ring may comprise a removable, interchangeable inner ring comprising one or more biometric sensors.

Referring now to the exploded perspective view of FIG. 1B, components of wearable device 100 are depicted. One or more input devices 102 is shown along the annular structure, and the one or more input devices may include a touch sensor, a sound sensor, a motion sensor, and a button. One or more output devices 104 is shown along the annular structure, and the one or more output devices may include a haptic source, a sound source, and an electromagnetic radiation source. The electromagnetic radiation source may transmit signals over a BT protocol, over a BTLE protocol, over a Near-Field Communication (NFC) protocol, over Wi-Fi, over a mesh network (for example Thread, Zigbee, Z-Wave, and the like), over ultra-wideband, over RF (Radio Frequency), over IR (Infrared), over cellular communications (including 2G, 3G, 4G/LTE, LTE-M/LTE-Cat-M, NB-IoT, and the like), or over the Global Positioning System (GPS). One or more antenna assemblies 113 and one or more wireless communication units 115 are shown along the annular structure. A processor 119, memory 121, power storage device 123, and an energy harvesting device 125 are shown along the annular structure.

Referring now to the exploded perspective view of FIG. 1C, one or more biometric sensors 127 are shown on at least one region of the inner circumferential surface of the annular structure. In this embodiment, the one or more biometric sensors 127 is configured on the inner circumferential surface of first ring 131. In this embodiment, first ring 131 is configured to share a center with second ring 133, and second ring 133 is removable from first ring 131. In this embodiment, first ring 131 is proximal to the center and second ring 133 is distal from the center, i.e., first ring 131 is nested within second ring 133. In this embodiment, first ring 131 may be considered an inner ring, and the inner ring may be interchangeable (i.e., inner rings with different biometric sensors may be exchanged). In some embodiments, the wearable device may include a tactile surface, including a raised or recessed contoured surface, configured to orient the wearable device with respect to a user's finger. In some embodiments, this tactile surface may be placed on the button itself to both locate the button as well as orient the ring without having to look at the ring. This is especially helpful for people with low vision or no vision (blind) and also for everyone in low light (ex: at night) or in the dark (ex: when in bed).

In certain embodiments and as depicted in FIGS. 2A and 2B, second ring 223 is a power storage device and is a removable component of wearable device 200, i.e. it can be detached from first ring 201. As shown in FIG. 2A, second ring 223 is configured to attach to first ring 201 in order to supply stored electric power to components of wearable device 200. In this embodiment, the power storage device is removable and interchangeable; once the power storage device is depleted of its charge, it may be detached and replaced with a fully charged power storage device. A user does not need to remove the wearable device when the battery is depleted, but can simply exchange the depleted battery for a fully charged battery.

A user may continue to use the wearable device while the depleted power storage device is charging at a charging station configured to transmit electric power to one or more power storage devices using wired or wireless charging. The wearable device may include a power storage device indicator light configured on the outer circumferential surface and configured to emit visible light in order to alert a user that the power storage device requires recharging. The power storage device may be any suitable type of battery, such as lithium ion, nickel cadmium, and the like.

Thus, the wearable device may comprise a first ring and a second ring, wherein the first ring and the second ring are configured to share a center. In an embodiment, the first ring is removable from the second ring. In another embodiment, the first ring is adjacent to the second ring, and the first ring comprises the power storage device.

The removable first ring may attach to the second ring magnetically. The first and second rings may be configured with two or more contacts to avoid rocking of the interlocking first and second rings. At minimum, contacts between the interlocking first and second rings must include power and ground. The distance between the contacts may be varied, including adjacent to each other, at opposite sides of the ring from each other, 120 degrees apart from each other (e.g., for 3 contacts), or in any other configuration depending on design complexity and minimizing the risk of electrical shorting. The wearable device may be configured to be waterproof.

In certain embodiments and as depicted in FIGS. 2C and 2D, second ring 223 and third ring 225 are power storage devices and are removable components of wearable device 210, i.e. they can be detached from first ring 201. As shown in FIG. 2C, second ring 223 and third ring 225 are configured to attach to first ring 201 in order to supply stored electric power to components of wearable device 210. In this embodiment, the power storage devices are removable; when one power storage device is depleted of its charge, it may be detached and recharged without loss of function of the wearable device because the other power storage device is still attached. A user does not need to remove the wearable device when one power storage device is depleted, but can simply remove the depleted power storage device and continue to use the wearable device powered by the remaining power storage device. In some embodiments, this detachable power storage device may attach magnetically for ease of removal and attachment.

In the embodiment of FIGS. 2A and 2B, button 208 is an input device provided on the outer circumferential surface, and electromagnetic radiation source 206 is an output device provided on the outer circumferential surface. A user may briefly press button 208 or press and hold button 208 for a predetermined amount of time to supply a touch input. In some embodiments, the button may be sub-flush, proud, or flush relative to the adjacent surface. Electromagnetic radiation source 206 may be configured to emit electromagnetic radiation signals, including invisible light signals such as infrared radiation (“IR”).

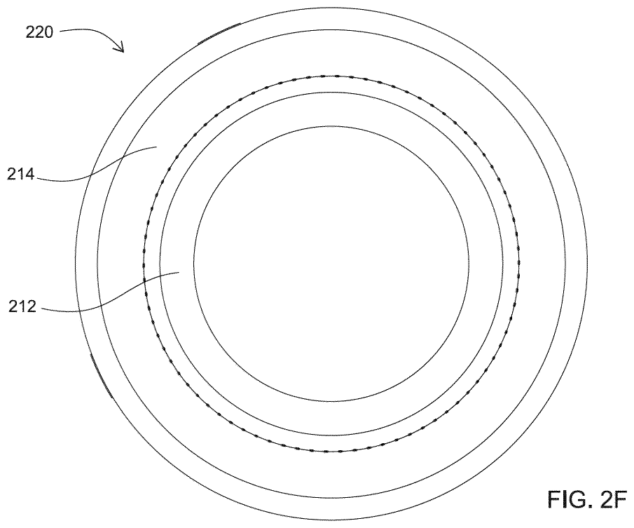

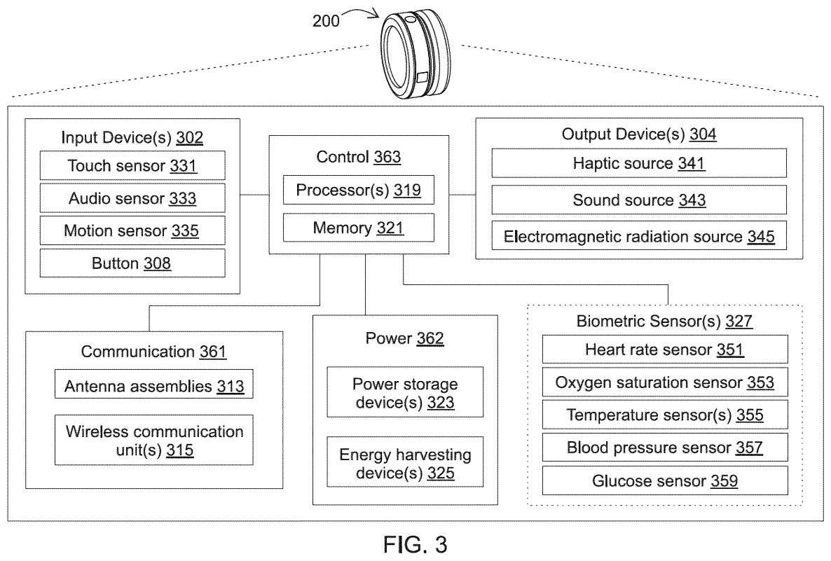

FIG. 3 is a block diagram of a wearable device according to various embodiments of the present disclosure. Exemplary wearable device 200 is shown having components such as a control unit 363, one or more input devices 302, one or more output devices 304, a power unit 362, a communication unit 361, and one or more biometric sensors 327. It is understood that implementing all of the illustrated components is not required, and that fewer or greater components may alternatively be implemented.

In more detail, the control unit 363 typically functions to control overall operations of the wearable device 200, in addition to the operations associated with application programs stored in the memory 321. The one or more processors 319 may control one or more functions of the wearable device by processing data, information, signals, and the like, or activating application programs. The memory 321 may be configured to store instructions and application programs (or applications) executable by the one or more processors 319 of the wearable device 200, data or instructions for operations of the wearable device 200, and the like. Some application programs may be downloaded from an external server or network via wireless communications, and others may be installed at the time of manufacturing or shipping of the wearable device. It is common for application programs to be stored in the memory 321, installed in the wearable device 200, and executed by one or more processors 319 to perform an operation or function.

The one or more processors 319 may include any suitable processing unit capable of receiving data as input, processing the inputted data in accordance with computer-executable instructions, and generating data as output. The one or more processors 319 may include any suitable processing unit including, but not limited to, a microprocessor, a central processing unit, a microcontroller, a Reduced Instruction Set Computer (RISC) microprocessor, an Application Specific Integrated Circuit (ASIC), a Complex Instruction Set Computer (CISC) microprocessor, a System-on-a-Chip (SoC), a Field-Programmable Gate Array (FPGA), a digital signal processor (DSP), etc. The microarchitecture of the one or more processors 319 may be designed to support any of a variety of computer-executable instructions, and may include any number of constituent components, including multiplexers, registers, arithmetic logic units, branch predictors, cache controllers for controlling read/write operations to cache memory, etc. Components of wearable device 200 may be configured on a printed circuit board (PCB), including a flex PCB, a rigid-flex PCB, and a rigid PCB.

In more detail, the one or more input devices 302 may include a touch sensor 331, an audio sensor 333, a motion sensor 335, and a button 308. Data is obtained by the one or more input devices 302 and may be analyzed and processed according to user commands. The touch sensor may include a fingerprint sensor, a pressure sensor, or a touch sensor. The fingerprint sensor may be configured on at least one region of the inner circumferential surface, and the pressure sensor and the button may be configured on the outer circumferential surface. The pressure or touch sensor may include a capacitive sensor array, including a row-column-mutual-cap architecture, a pixelated-self-cap architecture, and the like. The button, pressure or touch sensor, or the motion sensor may activate the fingerprint sensor or the electromagnetic radiation source, i.e., when the pressure sensor detects pressure or the motion sensor detects movement.

The sound sensor may include one or more microphones. The one or more microphones may comprise a first microphone configured to detect sounds and a second microphone to detect sounds, wherein the first microphone and the second microphone are configured for active noise cancellation.

The wearable device further comprises an accelerometer, a gyroscope, and a magnetometer, and the motion sensor is configured to receive information from the accelerometer, the gyroscope, and the magnetometer. The accelerometer may be a piezoelectric accelerometer, including high impedance or low impedance piezoelectric accelerometers, configured to detect and calculate shaking, tilting, swinging, rotating, and the like. The gyroscope may be a piezoelectric gyroscope configured to detect angular rotational velocity and acceleration. The magnetometer may detect a magnetic field or magnetic dipole moment to measure the direction, strength, and relative change of the magnetic field. The motion sensor 335 and one or more processors 319 may be configured to calculate acceleration, velocity, linear acceleration along x, y, or z axes, and rotational displacement about x, y, or z axes.

In more detail, the one or more output devices 304 may include a haptic source 341, a sound source 343, and an electromagnetic radiation source 345. The haptic source 341 may be configured to generate haptic signals, and may generate haptic signals when the button is pressed or pressed and held. The haptic source 341 may be configured to generate haptic signals when the wearable device is pointed at a valid receiver device, with or without a user pressing the button or pressing and holding the button. The haptic source 341 may be configured to generate haptic signals of increasing frequency as the user moves closer to the valid receiver device, thereby enabling orientation and navigation for blind or visually impaired users.

The sound source 343 may include a speaker assembly configured to generate sound signals. The electromagnetic radiation source 345 may be configured to generate one or more electromagnetic radiation signals. The electromagnetic radiation source may comprise a light emitting diode (LED), and the electromagnetic radiation signals may include one or more visible light signals and one or more invisible light signals. The one or more invisible light signals may include infrared (IR) radiation or radio frequency (RF) radiation.

In more detail, the power unit 362 may include one or more power storage devices 323 and one or more energy harvesting devices 325. The one or more power storage devices 323 may be configured to store electric power, and the one or more energy harvesting devices 325 may be configured to transmit electric power to the one or more power storage devices 323. The one or more energy harvesting devices 325 may use any suitable harvesting approach, including a thermoelectric generator (TEG) using the Seebeck effect, a piezoelectric element using motion, a transducer using gravity, a biofuel cell using perspiration, and the like, to passively recharge the one or more power storage devices 323.

In more detail, the communication unit 361 may include one or more antenna assemblies 313 and one or more wireless communication units 315. The communication unit 361 permits communications such as wireless communications between the wearable device 200 and one or more receiver devices, wireless communications between the wearable device 200 and one or more smart device receivers, wireless communications between the wearable device 200 and one or more hubs (including smart devices, charging stations, and mobile devices) configured to interface with the internet, and wireless communications between the wearable device 200 and at least one network. The one or more antenna assemblies 313 may be configured on the outer circumferential surface to facilitate wireless communications. The communication unit 361 may be configured to use received signal strength indicator (RSSI), received channel power indicator (RCPI), or ping latency in milliseconds, including for location positioning.

The one or more wireless communication units 315 may be configured for wireless communication over a Bluetooth (BT) protocol, over a Bluetooth Low Energy (BTLE) protocol, over an NFC protocol, over Wi-Fi, over a mesh network (for example Thread, Zigbee, Z-Wave, and the like), over ultra-wideband, over RF, over IR, over cellular communications, or over GPS. The NFC protocol may be configured to scan for nearby devices at time intervals or to scan when motion sensor 335 detects movement.

In more detail, the one or more biometric sensors 327 may include a heart rate sensor 351, an oxygen saturation sensor 353, one or more temperature sensors 355, a blood pressure sensor 357, and a glucose sensor 359. The biometric sensors 327 may be configured on the inner circumferential surface, may be configured for relative measurements, and may require calibration for absolute measurements. The one or more temperature sensors 355 may include a first temperature sensor on at least one region of the inner circumferential surface and configured to sense a user's temperature, and a second temperature sensor on at least one region of the outer circumferential surface and configured to sense an ambient temperature. The one or more temperature sensors 355 may include contact or non-contact temperature sensors (e.g., optical) and may be configured to convert measured temperature to a user's internal body temperature. The wearable device may be configured with more than one biometric sensor to obtain accurate biometric data and reduce the number of false negatives and false positives.

The wearable device 200 may be configured so that the memory 321 stores instructions executable by the one or more processors 319, which when executed cause the wearable device to detect sounds, recognize trigger words, recognize speech commands, and communicate with one or more voice assistants. In certain embodiments, the wearable device 200 is configured with a firmware/software voice wrapper to enable interoperability with all voice assistants, for example Alexa, Siri, Cortana, Google, and the like, from a single wearable device. This obviates the requirement for separate voice transmitters for each voice transmitter (e.g., separate Alexa transmitter, separate Siri transmitter, separate Google transmitter, etc.) In certain embodiments, a user may press and hold the button to activate the sound sensor to enable the user to provide voice commands to voice assistants.

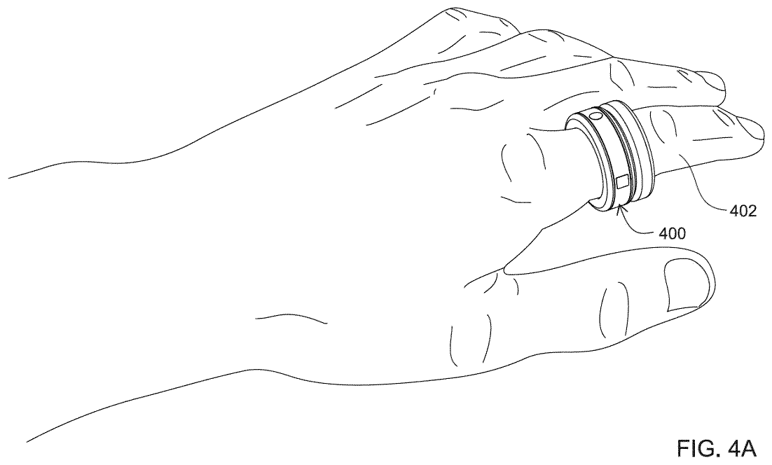

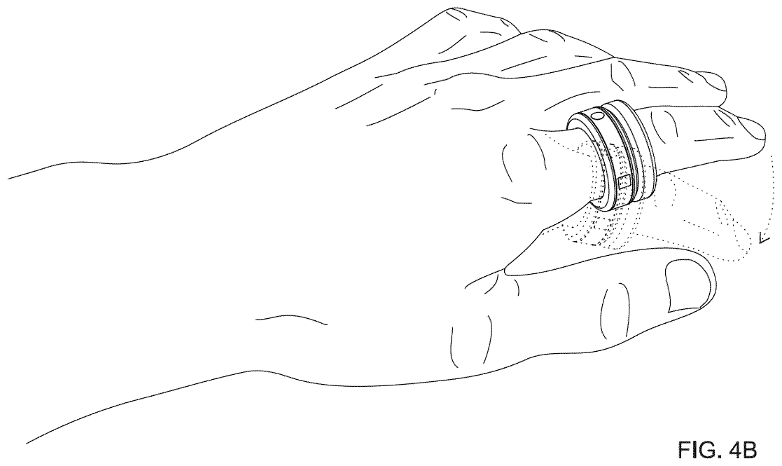

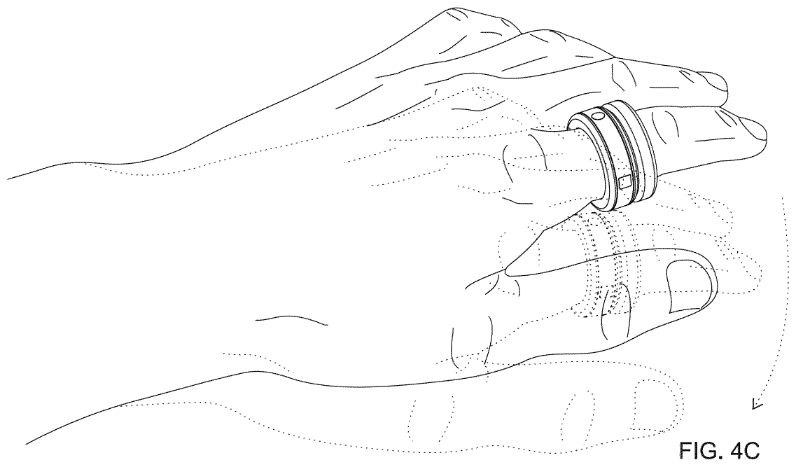

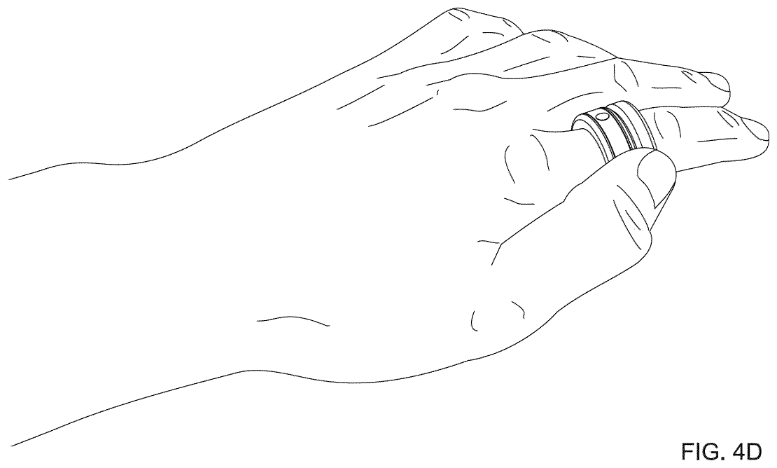

As depicted in the exemplary embodiment of FIG. 4A, a user may install wearable device 400 on a finger 402 by inserting the finger into the ring-shaped body. The wearable device 400 may be embodied as a ring configured to be disposed on any of the user's fingers (i.e., placed on and surrounding a user's finger), including a user's finger adjacent to a user's thumb for the most ergonomic use. As shown in FIGS. 4B and 4C, a user may use muscles of the hands to perform various motions. As depicted in FIG. 4B, a user moves a finger by activating smaller muscles of the hands (i.e., fine motor control—most ergonomic and least energy intensive). As depicted in FIG. 4C, a user moves a hand by activating larger muscles of the hands (i.e., gross motor control—less ergonomic and more energy intensive). The motion sensor of the wearable device may detect motion of a finger, motion of a hand, and motion of a user's body. The wearable device is configured to receive data from the motion sensor as input, process the inputted data in accordance with computer-executable instructions, and generate data corresponding to the gesture or motion as output. As depicted in FIG. 4D, a user may activate a touch or pressure sensor or a button, with a finger, including an adjacent finger (e.g., a thumb).

### Wearable Device and Receiver Systems

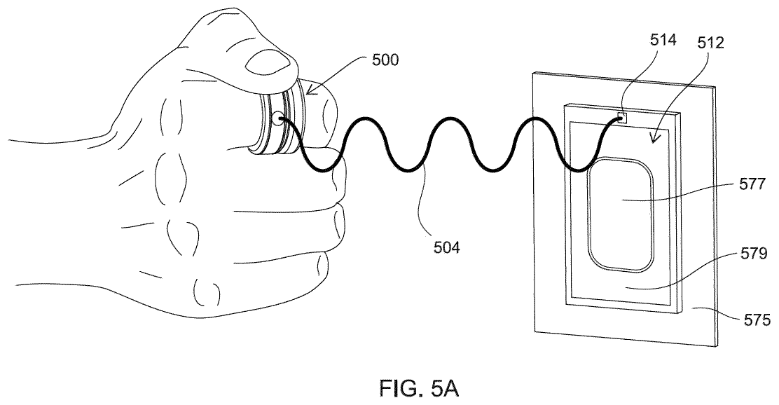

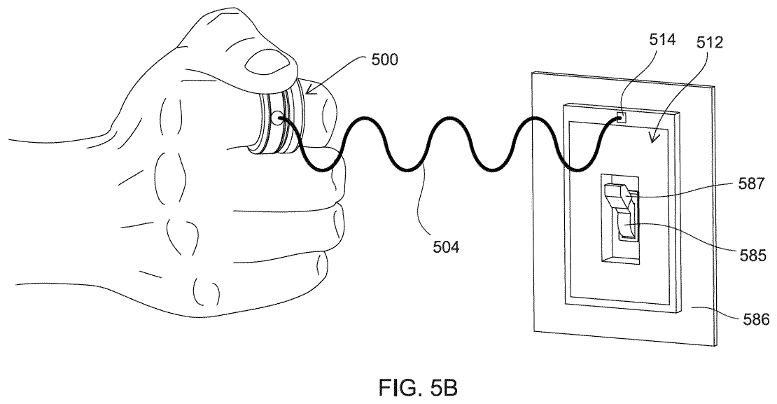

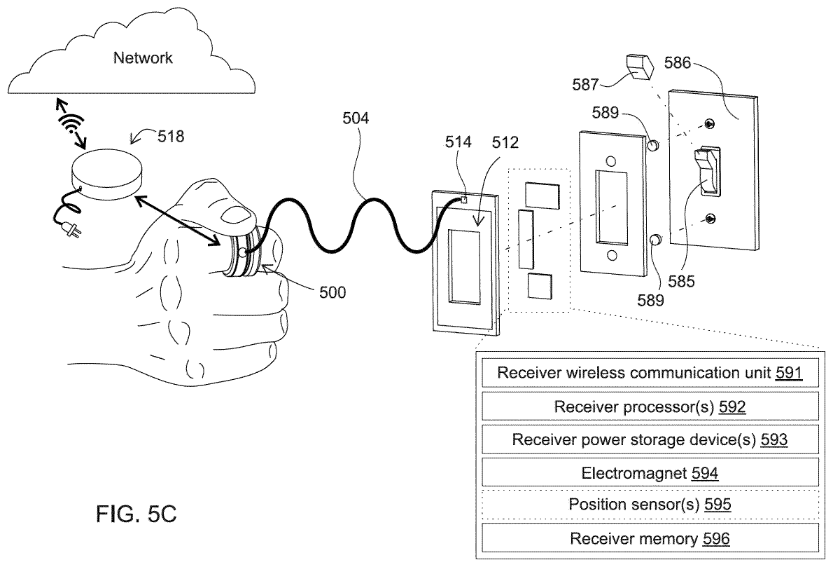

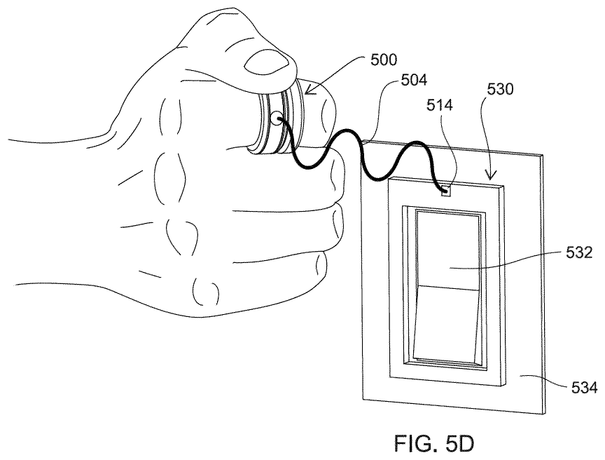

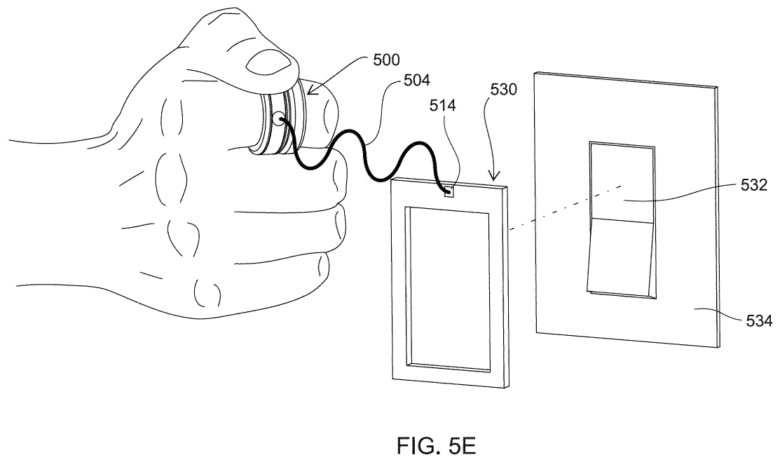

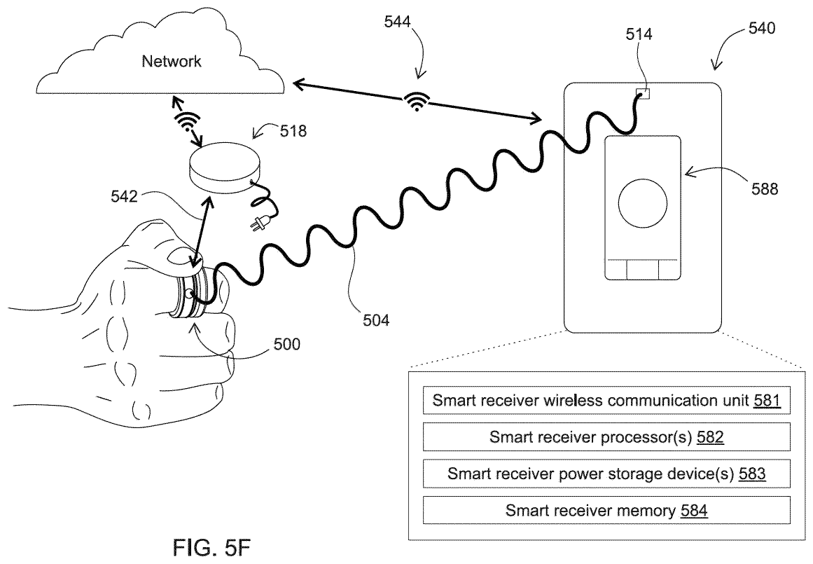

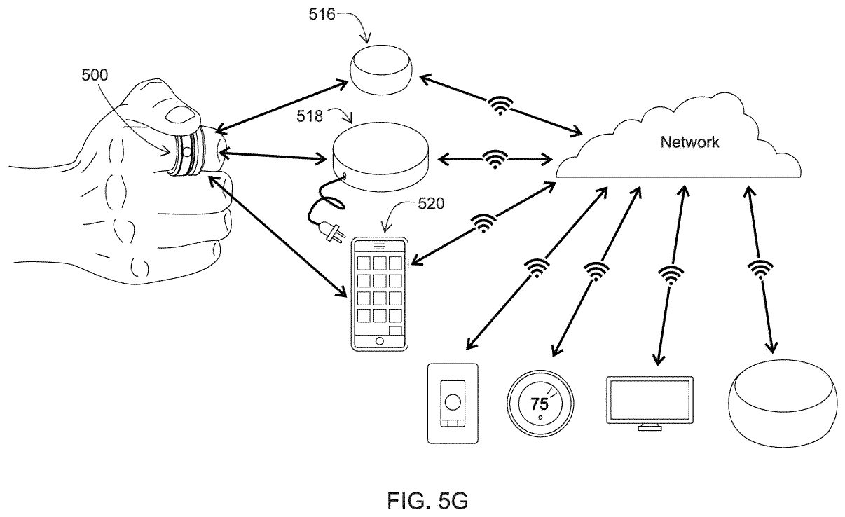

As depicted in the exemplary embodiments of FIG. 5A, FIG. 5B, FIG. 5C, FIG. 5D, FIG. 5E, FIG. 5F, and FIG. 5G, the wearable devices disclosed herein may be incorporated into systems comprising one or more receiver devices, one or more smart receiver devices, and one or more hubs and smart devices. The wearable devices may be configured to communicate with the one or more receiver devices, the one or more smart receiver devices, and the one or more hubs and smart devices.

In FIG. 5A, wearable device 500 may be embodied as a ring configured to be disposed on a user's finger and communicate with one or more receiver devices 512. Electromagnetic radiation signal 504 may be emitted from the electromagnetic radiation source of the wearable device and received by the electromagnetic radiation sensor 514 of the receiver device. Receiver device 512 may be installed on switch plate 575 of either a toggle light switch or a rocker light switch. Switch button 577 may be pressed manually to engage the light switch (i.e., to toggle either a toggle light switch or a rocker light switch). Switch button 577 may be coplanar, raised, or recessed relative to device faceplate 579.

In FIG. 5B, wearable device 500 may be embodied as a ring configured to be disposed on a user's finger and communicate with one or more receiver devices 512 installed on a toggle light switch. Electromagnetic radiation signal 504 may be emitted from the electromagnetic radiation source of the wearable device and received by the electromagnetic radiation sensor 514 of the receiver device. Receiver device 512 may be installed on switch plate 586 of a toggle light switch. Toggle switch 585 may be engaged manually and may be equipped with ferromagnetic switch extender 587 to enable magnetic control of the toggle switch using a hidden electromagnet. Ferromagnetic switch extender 587 may protrude past the receiver device and facilitate manual switching.

FIG. 5C shows an exploded perspective view of the receiver device of FIG. 5B, including a block diagram of the receiver device according to various embodiments of the present disclosure. The receiver device 512 is shown having components such as a receiver wireless communication unit 591, one or more receiver processors 592, one or more receiver power storage devices 593, an electromagnet 594, one or more position sensors 595, and receiver memory 596. In an alternative embodiment, receiver device 512 does not include one or more position sensors 595. In an alternative embodiment, wearable device 500 communicates wirelessly with both receiver device 512 and charging station 518, and charging station 518 communicates wirelessly with a network.

In more detail, the receiver wireless communication unit 591 permits communications such as wireless communications between the wearable device 500 and the receiver device 512. The receiver wireless communication unit 591 comprises a receiver electromagnetic radiation sensor configured to detect the electromagnetic radiation signal 504 emitted from the electromagnetic radiation source of the wearable device.

The one or more receiver processors 592 typically function to control overall operations of the receiver device 512, in addition to the operations associated with application programs stored in the receiver memory 596. The one or more receiver processors 592 may control one or more functions of the receiver device by processing data, information, signals, and the like, or activating application programs. The receiver memory 596 may be configured to store instructions and application programs (or applications) executable by the one or more processors 592, data or instructions for operations of the receiver device 512, and the like. Some application programs may be downloaded from an external server or network via wireless communications, and others may be installed at the time of manufacturing or shipping of the receiver device. It is common for application programs to be stored in the receiver memory 596, installed in the receiver device 512, and executed by one or more receiver processors 592 to perform an operation or function.

The one or more receiver processors 592 may include any suitable processing unit capable of receiving data as input, processing the inputted data in accordance with computer-executable instructions, and generating data as output. The one or more receiver processors 592 may include any suitable processing unit including, but not limited to, a microprocessor, a central processing unit, a microcontroller, a Reduced Instruction Set Computer (RISC) microprocessor, an Application Specific Integrated Circuit (ASIC), a Complex Instruction Set Computer (CISC) microprocessor, a System-on-a-Chip (SoC), a Field-Programmable Gate Array (FPGA), a digital signal processor (DSP), etc. The microarchitecture of the one or more receiver processors 592 may be designed to support any of a variety of computer-executable instructions, and may include any number of constituent components, including multiplexers, registers, arithmetic logic units, branch predictors, cache controllers for controlling read/write operations to cache memory, etc. Components of receiver device 512 may be configured on a printed circuit board (PCB), including a flex PCB, a rigid-flex PCB, and a rigid PCB.

The one or more receiver power storage devices 593 may be configured to store electric power. The one or more receiver power storage devices 593 may be removable, rechargeable, and interchangeable. The one or more power storage devices 593 may be of any suitable type of battery, such as lithium ion, nickel cadmium, and the like. The electromagnet 594 may comprise a solenoid, a servomotor, a stepper motor, motor, or the like. An alternative embodiment of the receiver device may include one or more limit switches and/or one or more position sensors 595 that may comprise a Hall effect sensor configured to detect a state of electromagnet 594 by the presence and magnitude of a magnetic field. The one or more position sensors may be magnetic, optical, inductive, and the like and may be linear or rotary. The receiver device 512 may be configured so that the receiver memory 596 stores receiver instructions executable by the one or more receiver processors 592, which when executed cause the receiver device 512 to detect a position of electromagnet 594 using the one or more position sensors 595, actuate electromagnet 594, and reconfigure the position. An alternative embodiment of the receiver device 512 may also comprise a receiver electromagnetic radiation source configured to generate one or more receiver electromagnetic radiation signals that may be detected by one or more wearable devices comprising an electromagnetic radiation sensor.

As shown in FIG. 5C, receiver device 512 may be installed over a toggle light switch 585 and switch plate 586, including by magnetic force resulting from magnets 589. The light switch 585 may protrude past the receiver device cavity where electromagnet 594 and the one or more position sensors 595 are configured to enable reconfiguration of the light switch position. The light switch 585 may be equipped with ferromagnetic light switch extender 587 to enable magnetic control of the toggle switch using a hidden electromagnet. Ferromagnetic switch extender 587 may protrude past the receiver device and remain accessible for manual switching. The light switch 585 may be covered as in FIG. 5A and configured so that switch button 577 may be pressed manually to engage the light switch, e.g., a toggle switch or a rocker switch.

A user may point a finger bearing wearable device 500 and activate the wearable device to emit electromagnetic radiation signal 504, including IR signals. The receiver device 512 installed on switch plate 586 detects electromagnetic radiation signal 504 using receiver wireless communication unit 591, one or more receiver processors 592 receives electromagnetic radiation signal 504 as input, and one or more receiver processors 592 processes electromagnetic radiation signal 504 in accordance with computer-executable instructions stored in receiver memory 596. When executed, the receiver instructions may cause the receiver device 512 to detect a position of electromagnet 594 and light switch 585 using the one or more position sensors 595, actuate electromagnet 594, and reconfigure the position of electromagnet 594 and light switch 585. The receiver device may emit one or more receiver electromagnetic radiation signals to communicate information wirelessly back to wearable device 500.

As shown in FIG. 5D, receiver device 530 may be installed over a rocker light switch 532 and switch plate 534, including by magnetic force. The rocker light switch 532 may protrude through receiver device 530 so that a user may manually engage rocker light switch 532. FIG. 5E shows an exploded perspective view of the receiver device of FIG. 5D.

In some embodiments, receiver devices may be installed on various light switches, including toggle switches, rocker switches, rotating knob switches, side switches, sliders, push-button switches, single-pole switches, multi-location switches, dimmer switches, programmable timer switches, and the like. In some embodiments, receiver devices may be installed on doors, including residential, commercial, automobile, train, boat, airplane, etc. doors, to enable a user to open a door. In some embodiments, receiver devices may be installed on windows, including residential, commercial, automobile, train, boat, etc. doors, to enable a user to open a window. In some embodiments, receiver devices may be installed on window coverings, including curtains, shades, blinds, etc., to enable a user to open a window covering. In some embodiments, receiver devices may be installed on faucets, knobs, flush push buttons, flush handles, and the like. In some embodiments, receiver devices may be installed on wall sockets/plugs to control an internal relay that controls the flow of AC power from the wall into the device plugged in. In some embodiments, the user may control a receiver device, for example using input devices such as the touch sensor, the sound sensor, the motion sensor, and the button, in its line of sight using IR to control objects from a distance. In some embodiments, the user may control a receiver device, for example using input devices such as the touch sensor, the sound sensor, the motion sensor, and the button, not in its line of sight using BT, BTLE, or a mesh network to control objects from a distance.

In FIG. 5F, wearable device 500 may be embodied as a ring configured to be disposed on a user's finger and communicate with one or more smart receiver devices 540 installed on one or more smart devices 588 and communicate with one or more hubs, wherein the one or more hubs is configured to interface with a network, including the internet, and the network is configured to interface with the one or more smart devices 588. The one or more hubs may comprise a charging station 518. Electromagnetic radiation signal 504 may be emitted from the electromagnetic radiation source of the wearable device and received by the smart receiver wireless communication unit 581 of the smart receiver device.

In some embodiments, one or more smart receiver devices is configured to communicate with one or more wearable devices. The one or more smart receiver devices 540 may transmit a smart receiver identification (retrieved from smart receiver memory 584) by an electromagnetic radiation signal emitted by smart receiver wireless communication unit 581. In an embodiment, the one or more smart receiver devices 540 may transmit a smart receiver identification (retrieved from smart receiver memory 584) by infrared. In an embodiment, the one or more smart receiver devices 540 may transmit a smart receiver identification (retrieved from smart receiver memory 584) by BT or BTLE. Wearable device 500 may receive the smart receiver identification, execute instructions to retrieve from a lookup table output values corresponding to the smart receiver identification input value, and cause the one or more smart devices to perform one or more functions. Retrieval and execution of instructions from the lookup table may be performed by the wearable device memory or in the network. Once the identity of the smart device that the user seeks to control has been determined, the appropriate smart device's API may be invoked in the network. The system comprising one or more wearable devices and one or more smart receiver devices may be configured so that one or more smart receiver devices may be attached to one or more existing smart devices without expensive and time-consuming retrofitting.

FIG. 5F shows a block diagram of the smart receiver device according to various embodiments of the present disclosure. The smart receiver device 540 is shown having components such as a smart receiver wireless communication unit 581, one or more smart processors 582, one or more smart receiver power storage devices 583, and smart receiver memory 584. The smart receiver device 540 is shown installed on smart device 588, and smart device 588 communicates wirelessly with a network, for example using Wi-Fi 544. The wearable device 500 may communicate wirelessly with a network by emitting and receiving electromagnetic radiation signals 542.

In more detail, the smart receiver wireless communication unit 581 permits communications such as wireless communications between the wearable device 500 and the smart receiver device 540. The smart receiver wireless communication unit 581 comprises a smart receiver electromagnetic radiation sensor configured to detect the electromagnetic radiation signal 504 emitted from the electromagnetic radiation source of the wearable device.

The one or more smart receiver processors 582 typically function to control overall operations of the smart receiver device 540, in addition to the operations associated with application programs stored in the smart receiver memory 584. The one or more smart receiver processors 582 may control one or more functions of the smart receiver device by processing data, information, signals, and the like, or activating application programs. The smart receiver memory 584 may be configured to store instructions and application programs (or applications) executable by the one or more smart receiver processors 582, data or instructions for operations of the smart receiver device 540, and the like. Some application programs may be downloaded from an external server or network via wireless communications, and others may be installed at the time of manufacturing or shipping of the smart receiver device. It is common for application programs to be stored in the smart receiver memory 584, installed in the smart receiver device 540, and executed by one or more smart receiver processors 582 to perform an operation or function. The smart receiver memory 584 may store instructions executable by the one or more smart receiver processors 582 which when executed cause wearable device 500 to detect a smart receiver identification from smart receiver memory 584, communicate with a network, including the internet, and cause smart device 588 to perform one or more functions.

The one or more smart receiver processors 582 may include any suitable processing unit capable of receiving data as input, processing the inputted data in accordance with computer-executable instructions, and generating data as output. The one or more smart receiver processors 582 may include any suitable processing unit including, but not limited to, a microprocessor, a central processing unit, a microcontroller, a Reduced Instruction Set Computer (RISC) microprocessor, an Application Specific Integrated Circuit (ASIC), a Complex Instruction Set Computer (CISC) microprocessor, a System-on-a-Chip (SoC), a Field-Programmable Gate Array (FPGA), a digital signal processor (DSP), etc. The microarchitecture of the one or more smart receiver processors 582 may be designed to support any of a variety of computer-executable instructions, and may include any number of constituent components, including multiplexers, registers, arithmetic logic units, branch predictors, cache controllers for controlling read/write operations to cache memory, etc. Components of smart receiver device 540 may be configured on a printed circuit board (PCB), including a flex PCB and a rigid-flex PCB.

The one or more smart receiver power storage devices 583 may be configured to store electric power. The one or more smart receiver power storage devices 583 may be of any suitable type of battery, such as lithium ion, nickel cadmium, and the like.

In FIG. 5G, wearable device 500 may be embodied as a ring configured to be disposed on a user's finger and communicate with one or more hubs, wherein the one or more hubs is configured to interface with a network, including the internet, and the network is configured to interface with smart devices, including smart light switches, smart thermostats, smart televisions, and the like. The one or more hubs may comprise a smart device 516, a charging station 518, a mobile smart device 520, and the like.

The system depicted in FIG. 5G may be configured for payment processing. At a point of sale, wearable device 500 may communicate wirelessly, for example with a 13.56 MHz antenna, over a Bluetooth protocol (also referred to as Bluetooth), with a mobile phone to receive payment information, for example credit card information, debit card information, and the like. The wearable device may communicate wirelessly over Wi-Fi or a mesh protocol with a network, for example the internet, to receive payment information.

The devices and systems of the present disclosure may be used to facilitate navigation of a blind person in a space equipped with one or more receiver devices or one or more smart receiver devices. The wearable device may calculate location using the accelerometer (measuring steps) and the gyroscope (measuring angle) and communication between the wearable device and one or more receiver devices or smart receiver devices (using RSSI/RCPI or time-of-flight for IR or other electromagnetic sources). The one or more processors may compute polar coordinates (r and theta) to determine a user's location. The wearable device may use the GPS system to identify its location, and by pairing the wearable device to a mobile phone through Wi-Fi, RSSI, or Bluetooth, a user may compute location based on signal strength over the distance between the wearable device and the charging station 518.

The wearable device may be put into a scan mode so that it may guide a blind or visually impaired user on orientation and navigation. In scan mode, the wearable device 500 provides the user with haptic feedback when it is pointed toward a smart receiver device 540 or a receiver device 512. As the user moves toward the smart receiver device 540 or receiver device 512, the frequency of haptic feedback may increase. In this way the user may orient correctly (with the first haptic feedback) and navigate correctly (with subsequent haptic feedback increasing in frequency). Smart receiver device 540 and receiver device 512 may be attached to light switches which are generally located near doors, enabling a user to navigate both known spaces (e.g., a user's own home) or unknown spaces (e.g., an unfamiliar building). In known spaces, additional information such as number of steps, angle, and direction (obtained by the accelerometer, gyroscope, and magnetometer, respectively) may be used in conjunction with GPS, Wi-Fi, RSSI, Wi-Fi RCPI, or Wi-Fi latency to further increase accuracy of orientation and navigation.

The devices, systems, and methods of the present disclosure may be optimized for case of use for users with disabilities, including blind, deaf, non-verbal, loss of fine motor control (e.g., arthritic hands), loss of gross motor control, or mobility disability (e.g., wheelchair users, crutch users, walker users, cane users, etc.). The devices, systems, and methods eliminate the need for users to have internet or execute a costly, time-consuming retrofit of existing wall switches to smart switches while still permitting control of existing smart devices. The devices, systems, and methods also eliminate the need for users to install a smart speaker in every room to control light switches in every room. The devices, systems, and methods also eliminate the need for users to carry a smartphone at all times to control the home environment. The devices, systems, and methods also eliminate the need for users to pair every switch and every device, one by one, to smart speakers. The devices, systems, and methods eliminate the need for users to use an app to control such receiver devices, thereby allowing guests such as friends & family to use their own rings in one's home—leading to network effects. The devices, systems, and methods also eliminate the need for users to rewire, saving 11 hours/$2000 for a typical single-family home. The devices, systems, and methods also allow users to control Televisions natively, without the need for any external receiver components, by using Infrared.

The devices and systems of the present disclosure may require permissions for control and privacy to be set by an application on a mobile phone or through a web portal. This allows users to indicate which receivers are public (i.e., may be controlled by guests), which receivers are semi-public (i.e., may be controlled by trusted agents), and which receivers are private (i.e., may be controlled by the user only).

The devices and systems of the present disclosure may be configured by a user to enable remote monitoring and communication with a medical provider, for example a doctor or nurse. Distress signals may be transmitted by the wearable device to a medical provider (e.g., a registered nurse, nurse practitioner, or medical doctor) once the wearable device has detected inputs that have exceeded a pre-determined range customized for each user. For example, the motion sensor may detect a fall and the one or more biometric sensors may detect concerning vital signs (e.g., heart rate, oxygen, temperature, blood pressure, or glucose level exceeding a threshold set by a medical provider). The wearable device may transmit this information to a medical provider, who may contact the user, a family member, or a trusted agent to obtain more information, or may communicate with emergency services. The medical provider may monitor these data remotely and intervene as appropriate.

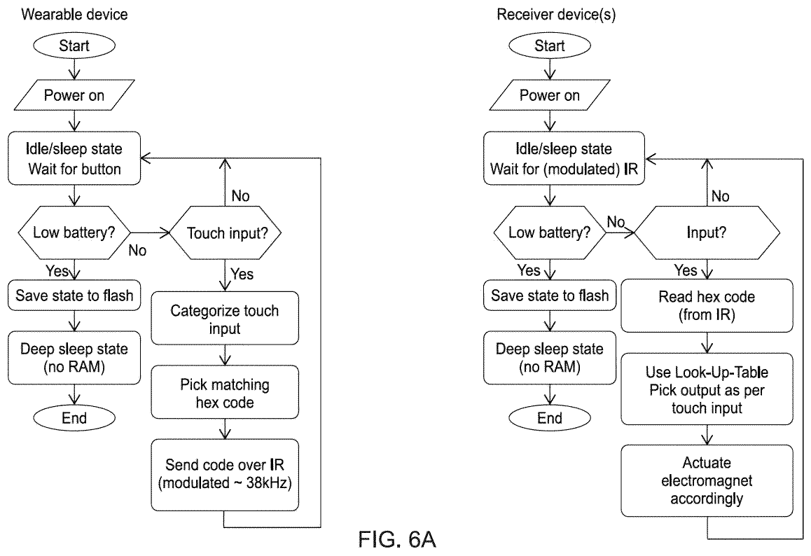

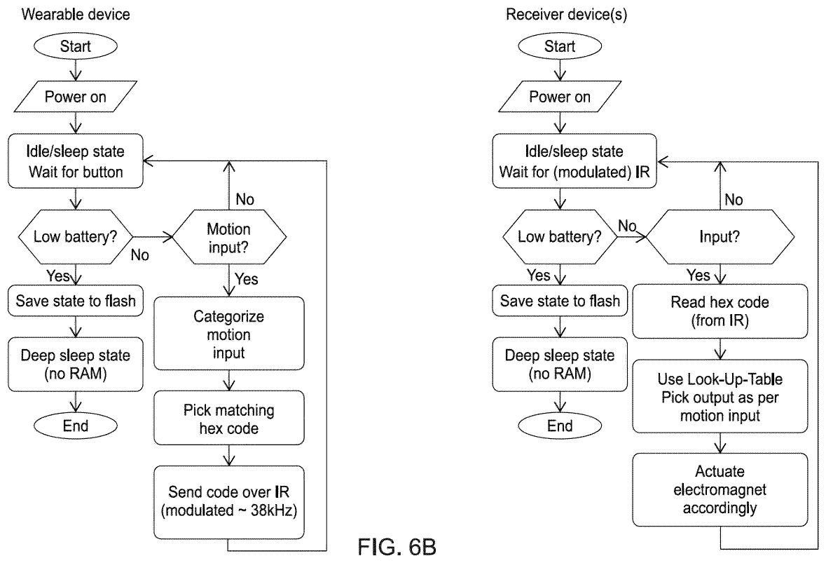

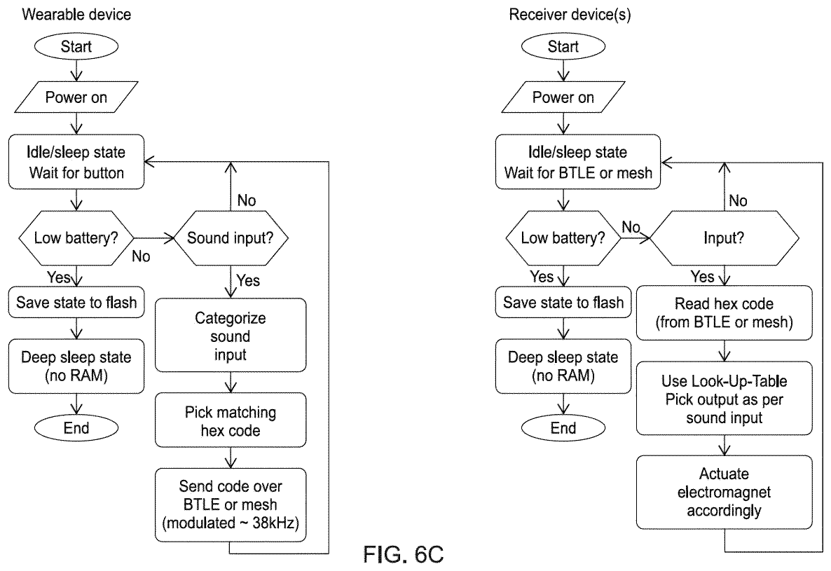

As depicted in the flowcharts of FIG. 6A, FIG. 6B and FIG. 6C, a wearable device of the present disclosure may be configured with instructions executable by one or more processors to control one or more receiver devices without requiring the internet. In FIG. 6A, a user may engage the touch sensor to activate the wearable device to control one or more receiver devices. In FIG. 6B, a user may engage the motion sensor to activate the wearable device to control one or more receiver devices. In FIG. 6C, a user may engage the sound sensor to activate the wearable device to control one or more receiver devices.

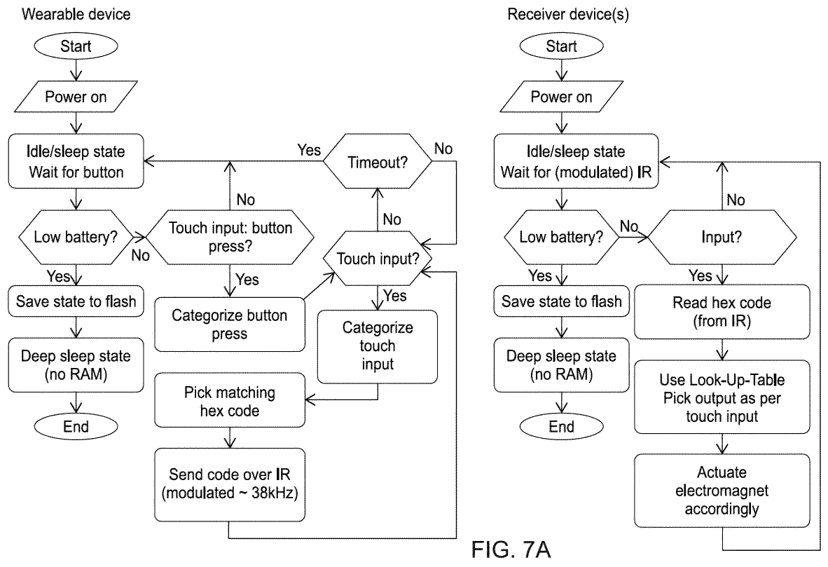

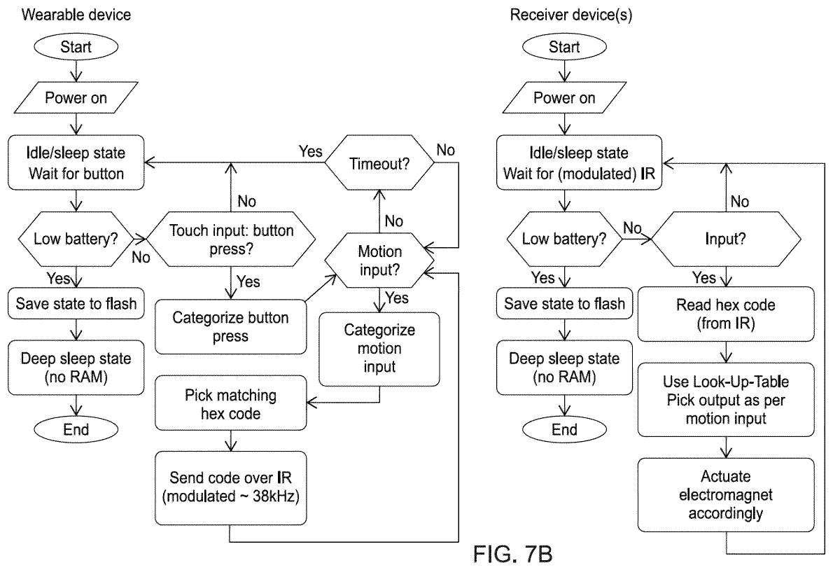

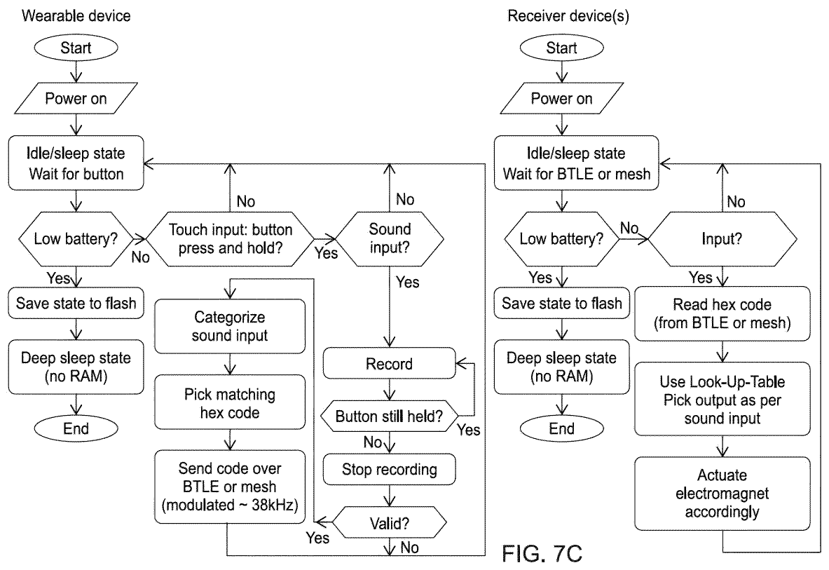

As depicted in the flowcharts of FIG. 7A, FIG. 7B and FIG. 7C, a wearable device of the present disclosure may be configured with instructions executable by one or more processors to control one or more receiver devices including button touch input without requiring the internet. In FIG. 7A, a user may both press the button and engage the touch sensor to activate the wearable device to control one or more receiver devices. In FIG. 7B, a user may both press the button and engage the motion sensor to activate the wearable device to control one or more receiver devices. In FIG. 7C, a user may both press the button and engage the sound sensor to activate the wearable device to control one or more receiver devices. This additional button press requirement may reduce the frequency of inadvertent activations of the wearable device.

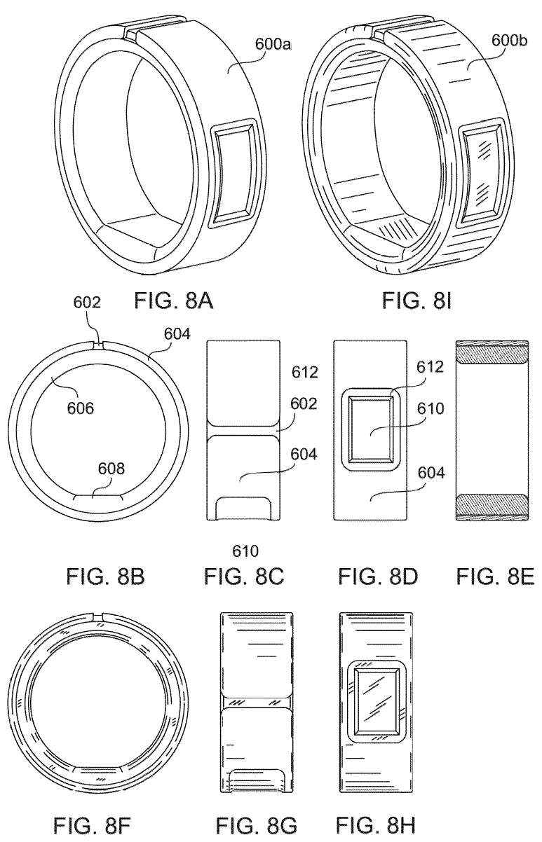

As depicted in FIGS. 8A-8D, an embodiment of the invention is presented in the form of a wearable device 600a. FIGS. 8F-8I disclose the same embodiment with surface shading. Here, the wearable device 600a is in its fully assembled state wherein a ring-shaped body comprises an inner circumferential surface 606 and an outer circumferential surface 604. One or more input, output, or combination input/output devices 608 may be provided on at least one region of the inner circumferential surface 606 of the annular structure of ring-shaped body of the wearable device 600a. One or more input devices and one or more output devices may be provided on at least one region of the outer circumferential surface 610 of the annular structure of ring-shaped body of the wearable device 600a. The device 610 may also be a combination input/output device. The device 610 may be elongated in a rectangular form within a bevel 612 for design or functional purposes. In the embodiment of FIGS. 8A-8D, device 610 is an input device provided on the outer circumferential surface, and electromagnetic radiation source 602 is an output device provided through a gap in the outer circumferential surface 604. A user may briefly activate device 610 or press and hold device 610 for a predetermined amount of time to supply a touch or button or combination input. In some embodiments, the device 610 may be sub-flush, proud, or flush relative to the adjacent surface. Electromagnetic radiation source 602 may be configured to emit electromagnetic radiation signals, including visible light signals (such as for button activation or battery indication) and including invisible signals such as infrared radiation.

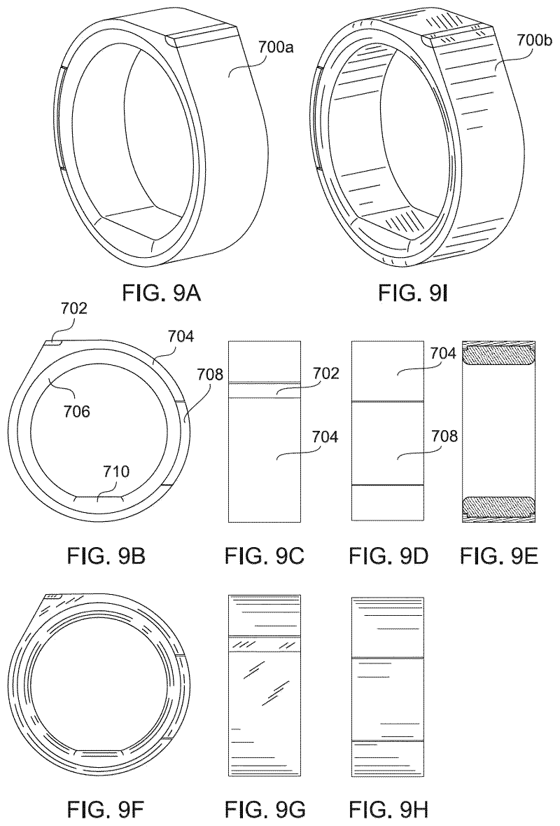

As depicted in FIGS. 9A-9D, an embodiment of the invention is presented in the form of a wearable device 700a. FIGS. 9F-9I disclose the same embodiment with surface shading. Here, the wearable device 700a is in its fully assembled state wherein a ring-shaped body comprises an inner circumferential surface 706 and an outer circumferential surface 704. One or more input, output, or combination input/output devices 710 may be provided on at least one region of the inner circumferential surface 706 of the annular structure of ring-shaped body of the wearable device 700a. One or more input devices and one or more output devices may be provided on at least one region of the outer circumferential surface 704 of the annular structure of ring-shaped body of the wearable device 700a. The device 708 may also be a combination input/output device. The device 708 may be elongated in a rectangular form for design or functional purposes. The wearable device may feature a pointed electromagnetic radiation source 702. In the embodiment of FIGS. 9A-9D, device 708 is an input device provided on the outer circumferential surface, and electromagnetic radiation source 702 is an output device provided where the outer circumferential surface 704 forms a protruding point. A user may briefly activate device 708 or press and hold device 708 for a predetermined amount of time to supply a touch or button or combination input. In some embodiments, the device 708 may be sub-flush, proud, or flush relative to the adjacent surface. Electromagnetic radiation source 702 may be configured to emit electromagnetic radiation signals, including visible light signals (such as for button activation or battery indication) and including invisible signals such as infrared radiation.

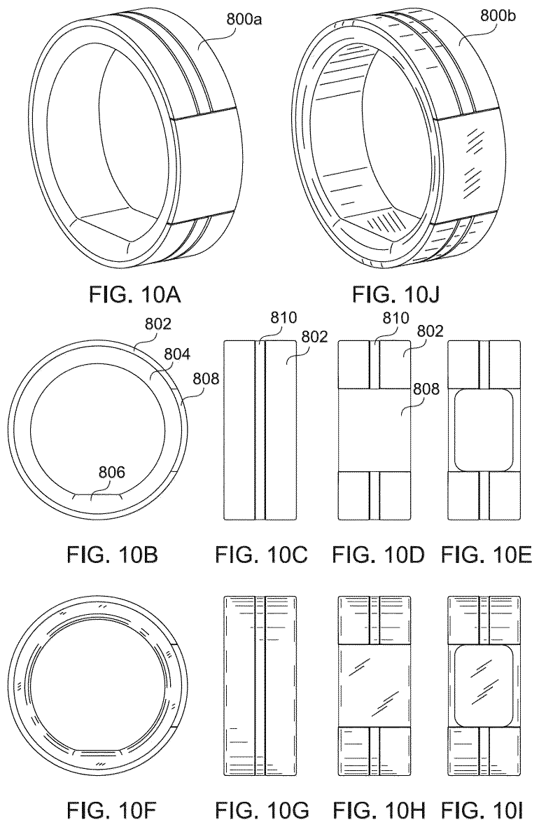

As depicted in FIGS. 10A-10E, an embodiment of the invention is presented in the form of a wearable device 800a. FIGS. 10F-9J disclose the same embodiment with surface shading. Here, the wearable device 800a is in its fully assembled state wherein a ring-shaped body comprises an inner circumferential surface 804 and an outer circumferential surface 802. One or more input, output, or combination input/output devices 806 may be provided on at least one region of the inner circumferential surface 804 of the annular structure of ring-shaped body of the wearable device 800a. One or more input devices and one or more output devices may be provided on at least one region of the outer circumferential surface 802 of the annular structure of ring-shaped body of the wearable device 800a. The device 808 may also be a combination input/output device. The device 808 may be elongated in a rectangular form for design or functional purposes. The wearable device 800a may feature a center groove around the outer circumference 810 for design or functional purposes. In the embodiment of FIGS. 10A-10D, device 808 is an input device provided on the outer circumferential surface. A user may briefly activate device 808 or press and hold device 808 for a predetermined amount of time to supply a touch or button or combination input. In some embodiments, the device 808 may be sub-flush, proud, or flush relative to the adjacent surface. Electromagnetic radiation source 810 may be configured to emit electromagnetic radiation signals, including visible light signals (such as for button activation or battery indication) and including invisible light signals such as infrared radiation.

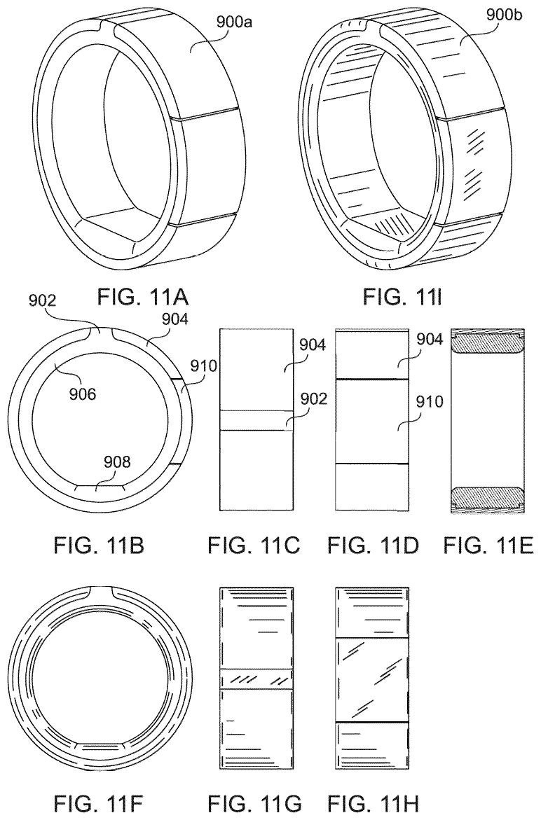

As depicted in FIGS. 11A-11D, an embodiment of the invention is presented in the form of a wearable device 900a. FIGS. 11F-11I disclose the same embodiment with surface shading. Here, the wearable device 900a is in its fully assembled state wherein a ring-shaped body comprises an inner circumferential surface 906 and an outer circumferential surface 904. One or more input, output, or combination input/output devices 908 may be provided on at least one region of the inner circumferential surface 906 of the annular structure of ring-shaped body of the wearable device 900a. One or more input devices and one or more output devices may be provided on at least one region of the outer circumferential surface 904 of the annular structure of ring-shaped body of the wearable device 900a. The device 910 may also be a combination input/output device. The device 910 may be elongated in a rectangular form for design or functional purposes. In the embodiment of FIGS. 11A-11D, device 910 is an input device provided on the outer circumferential surface, and electromagnetic radiation source 902 is an output device provided through a gap in the outer circumferential surface 904 such that the radiation source 902 is flush with the outers surface 904. A user may briefly activate device 910 or press and hold device 910 for a predetermined amount of time to supply a touch or button or combination input. In some embodiments, the device 910 may be sub-flush, proud, or flush relative to the adjacent surface. Electromagnetic radiation source 902 may be configured to emit electromagnetic radiation signals, including visible light signals (such as for button activation or battery indication) and including invisible light signals such as infrared radiation.

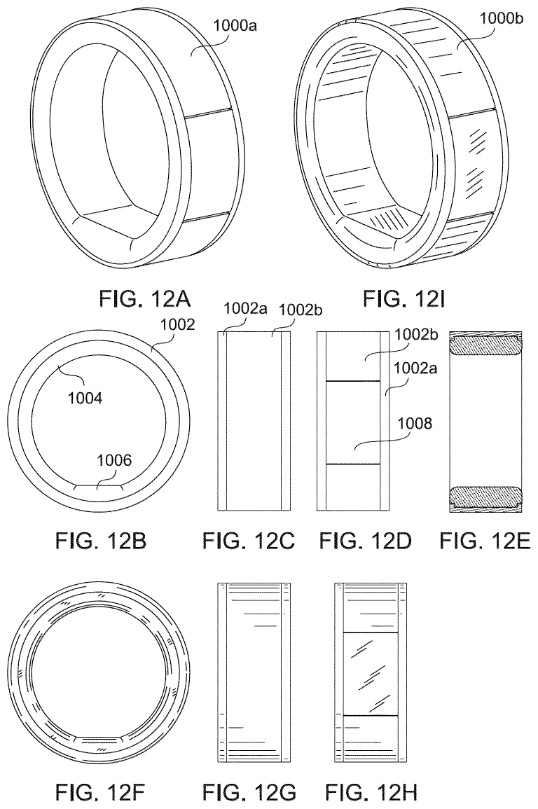

As depicted in FIGS. 12A-12F, an embodiment of the invention is presented in the form of a wearable device 1000a. FIGS. 12F-12I disclose the same embodiment with surface shading. Here, the wearable device 1000a is in its fully assembled state wherein a ring-shaped body comprises an inner circumferential surface 1004 and an outer circumferential surface 1002b. One or more input, output, or combination input/output devices 1006 may be provided on at least one region of the inner circumferential surface 1004 of the annular structure of ring-shaped body of the wearable device 1000a. One or more input devices and one or more output devices may be provided on at least one region of the outer circumferential surface 1002b of the annular structure of ring-shaped body of the wearable device 1000a. The device 1008 may also be a combination input/output device. The device 1008 may be elongated in a rectangular form for design or functional purposes. The wearable device 1000a may feature one or more border strips 1002a bordering the outer circumference 1002b for design or functional purposes. In the embodiment of FIGS. 12A-12D, device 1008 is an input device provided on the outer circumferential surface. A user may briefly activate device 1008 or press and hold device 1008 for a predetermined amount of time to supply a touch or button or combination input. In some embodiments, the device 1008 may be sub-flush, proud, or flush relative to the adjacent surface. Electromagnetic source 1002a may be configured to emit electromagnetic radiation signals, including visible light signals (such as for button activation or battery indication) and including invisible light signals such as infrared radiation.

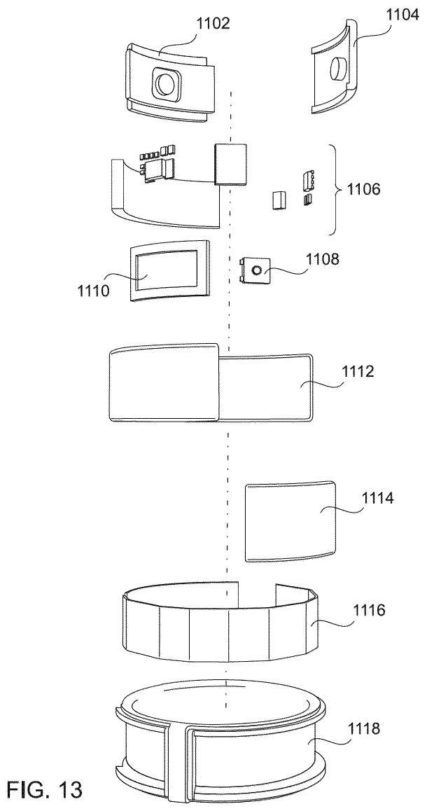

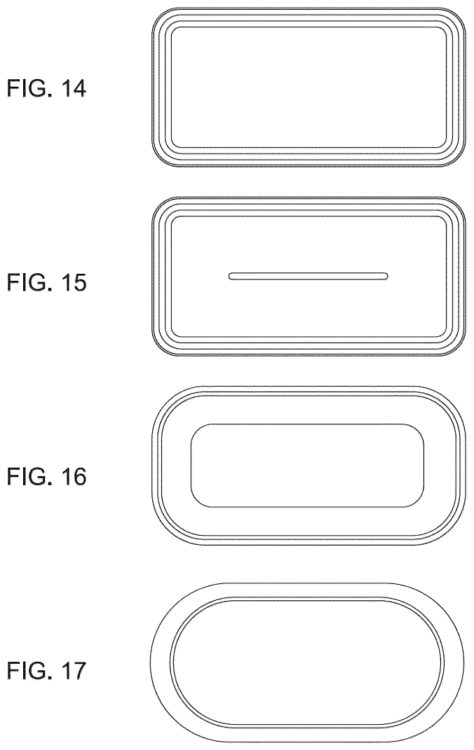

FIG. 13 is an exploded example of a wearable device invention disclosed herein. This exploded view depicts an inner button 1102 and outer button 1104, illustrated by device embodiments 610, 708, 808, 910, and 1008. The device embodiments are activated through electrical engineering components 1106, gasket 1110, and tac 1108. The invention features outer surfaces 1112, 1114, illustrated by device embodiments 604, 704, 802, and 904. The invention features an inner surface 1118, illustrated by device embodiments 606, 706, 804, 906, and 1004. The invention further features a flexible processor core 1116.

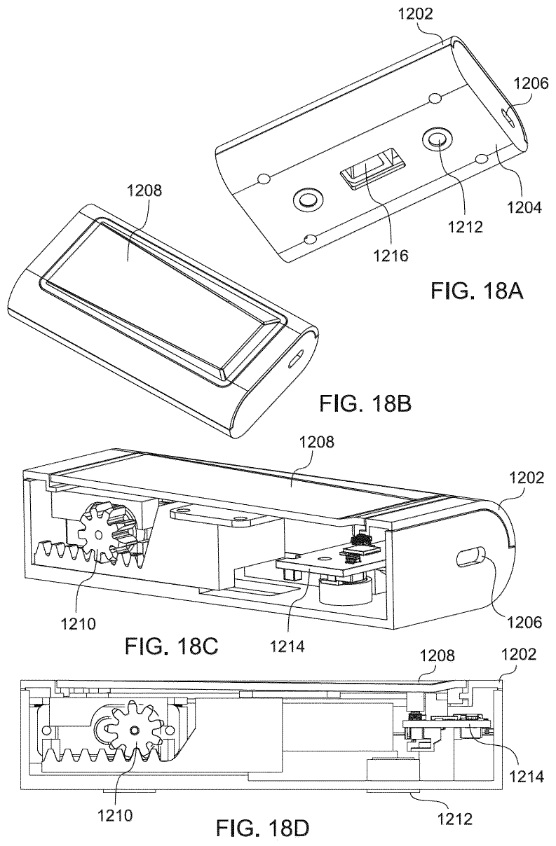

FIGS. 18A-18D illustrates a switch plate embodiment of the system invention designed to communicate with a wearable device ring. The switch plate may be comprised of a front housing 1202 and rear housing 1204 that may be affixed to a wall by means of one or more attachment points 1212 to accommodate common mounting application such as screws. A user facing button 1208 found in the front housing 1202 may control a light switch activated upon by a light switch port 1216 positioned in the rear housing. 1204. The light switch (not depicted) is activated on and off by means of a pinion gear system 1210 inside the housing. The pionion gear moves a n internal rack that envelopes a light switch (not depicted) that protrudes into the switch plate through the port 1216 when the switch plate is attached to a wall. An internal circuit board 1214 allows for remote communication with a wearable device ring embodiment described herein and activation by said ring.

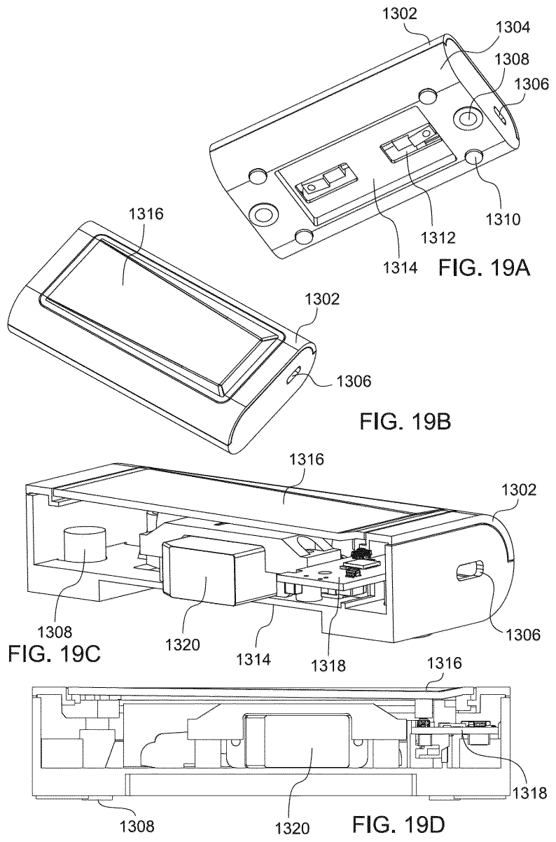

FIGS. 19A-19D illustrates a switch plate embodiment of the system invention designed to communicate with a wearable device ring. The switch plate may be comprised of a front housing 1302 and rear housing 1304 that may be affixed to a wall by means of one or more magnetic attachment points 1308 and buffered by one or more spacers 1310. A user facing button 1316 found in the front housing 1302 may control a light switch by pushing button 1316 to activate a tactile switch on the printed circuit board (PCB) 1318 that tells the servo 1320 to rotate a servo lever to contact the wall rocker switch. A user may also use a ring embodiment described herein to transmit an infrared signal through the front housing 1302 to an infrared receiver on the PCB 1318 to tell the servo 1320 to rotate the servo lever to contact the wall rocker switch.

### Numbered Embodiments

The following list of embodiments is not intended to be limiting and is included herein for illustrative purposes. The subject matter to be claimed is not limited to the following embodiments:

Embodiment 1. A wearable device comprising:
a. a ring-shaped body;
b. one or more input devices provided on at least one region of an inner circumferential surface or on at least one region of an outer circumferential surface; and
c. one or more output devices provided on at least one region of the inner circumferential surface or at least one region of the outer circumferential surface.

Embodiment 2. The wearable device of embodiment 1, wherein the one or more input devices is selected from the group consisting of a touch sensor, a sound sensor, a motion sensor, and a button.

Embodiment 3. The wearable device of embodiments 1 or 2, wherein the one or more output devices is selected from the group consisting of a haptic source, a sound source, and an electromagnetic radiation source.

Embodiment 4. The wearable device of any one of embodiments 1-3, further comprising one or more antenna assemblies, wherein the one or more antenna assemblies is configured on the outer circumferential surface.

Embodiment 5. The wearable device of any one of embodiments 1-4, further comprising one or more wireless communication units.

Embodiment 6. The wearable device of any one embodiments 1-5, further comprising one or more processors.

Embodiment 7. The wearable device of embodiment 6, wherein the one or more processors is configured to control one or more functions of the wearable device.

Embodiment 8. The wearable device of any one of embodiments 1-7, further comprising memory storing instructions executable by the one or more processors.

Embodiment 9. The wearable device of any one of embodiments 1-8, further comprising one or more power storage devices configured to store electric power.

Embodiment 10. The wearable device of embodiment 9, wherein the one or more power storage devices is removable and interchangeable.

Embodiment 11. The wearable device of embodiments 9 or 10, wherein the one or more power storage devices is configured to charge at a charging station.

Embodiment 12. The wearable device of embodiment 11, wherein the charging station is configured to transmit electric power to the one or more power storage devices. Embodiment 13. The wearable device of any one of embodiments 9-12, wherein the one or more power storage devices is configured for inductive or non-inductive wireless charging by pressure contacts, and wherein magnets are configured to align components for pressure contact.

Embodiment 14. The wearable device of any one of embodiments 9-13, further comprising one or more energy harvesting devices, wherein the one or more energy harvesting devices is configured to transmit electric power to the one or more power storage devices, and the one or more energy harvesting devices is a thermoelectric generator or a transducer.

Embodiment 15. The wearable device of any one of embodiments 1-14, further comprising one or more biometric sensors configured to sense and collect biometric information.

Embodiment 16. The wearable device of embodiment 15, wherein the one or more biometric sensors is selected from the group consisting of a heart rate sensor, an oxygen saturation sensor, a temperature sensor, a blood pressure sensor, and a glucose sensor.

Embodiment 17. The wearable device of embodiment 16, wherein the one or more biometric sensors comprises a first temperature sensor on at least one region of the inner circumferential surface and configured to sense a user's temperature, and a second temperature sensor on at least one region of the outer circumferential surface and configured to sense an ambient temperature.

Embodiment 18. The wearable device of any one of embodiments 2-17, wherein the touch sensor comprises a capacitive sensor, an inductive sensor, an optical sensor, or a combination thereof.

Embodiment 19. The wearable device of any one of embodiments 2-18, wherein the sound sensor comprises one or more microphones.

Embodiment 20. The wearable device of embodiment 19, wherein the one or more microphones comprises a first microphone configured to detect sounds and a second microphone to detect sounds, wherein the first microphone and the second microphone are configured for active noise cancellation.

Embodiment 21. The wearable device of any one of embodiments 8-20, wherein the memory stores instructions executable by the one or more processors, which when executed cause the wearable device to:
a. detect sounds;
b. recognize speech commands; and
c. communicate with one or more voice assistants.

Embodiment 22. The wearable device of any one of embodiments 2-21, wherein the wearable device further comprises an accelerometer, a gyroscope, and a magnetometer.

Embodiment 23. The wearable device of any one of embodiments 2-22, wherein the motion sensor is configured to receive information from the accelerometer, the gyroscope, and the magnetometer.

Embodiment 24. The wearable device of any one of embodiments 2-23, wherein the motion sensor and the one or more processors are configured to calculate displacement, velocity, acceleration, and rotational motion.

Embodiment 25. The wearable device of any one of embodiments 3-24, wherein the haptic source is configured to generate haptic signals.

Embodiment 26. The wearable device of any one of embodiments 3-25, wherein the sound source comprises a speaker assembly configured to generate sound signals.

Embodiment 27. The wearable device of any one of embodiments 3-26, wherein the electromagnetic radiation source is configured to generate one or more electromagnetic radiation signals.

Embodiment 28. The wearable device of any one of embodiments 3-27, wherein the electromagnetic radiation source comprises a light emitting diode.

Embodiment 29. The wearable device of embodiments 27 or 28, wherein the one or more electromagnetic radiation signals comprises one or more visible light signals or one or more invisible light signals.

Embodiment 30. The wearable device of embodiment 29, wherein the one or more invisible light signals comprises infrared radiation or radio frequency radiation.

Embodiment 31. The wearable device of any one of embodiments 5-30, wherein the one or more wireless communication units is configured for wireless communication over a Bluetooth protocol, over a Near-Field Communication protocol, over Wi-Fi, over a mesh network, over ultra-wideband, over radio frequency, over infrared, over cellular communication, or over the Global Positioning System.

Embodiment 32. The wearable device of embodiment 31, wherein the one or more wireless communication units is configured for wireless communication over a Bluetooth protocol or a mesh protocol with one or more hubs.

Embodiment 33. The wearable device of embodiment 32, wherein the one or more hubs is configured to interface with the internet.

Embodiment 34. The wearable device of embodiment 33, wherein the one or more hubs is selected from the group consisting of the charging station, a mobile device, and a smart device.

Embodiment 35. The wearable device of any one of embodiments 15-33, wherein the one or more biometric sensors is configured on the inner circumferential surface.

Embodiment 36. The wearable device of any one of embodiments 18-35, wherein the fingerprint sensor is configured on at least one region of the inner circumferential surface.

Embodiment 37. The wearable device of any one of embodiments 1-36, further comprising a tactile surface configured to orient the wearable device with respect to a user's finger.

Embodiment 38. The wearable device of any one of embodiments 1-37, further comprising a power storage device indicator light configured on the outer circumferential surface.

Embodiment 39. The wearable device of any one of embodiments 1-38, embodied as a ring comprising:
a. a first ring; and
b. a second ring;
c. wherein the first ring and the second ring are configured to share a center.

Embodiment 40. The wearable device of embodiment 39, wherein the first ring is removable from the second ring.

Embodiment 41. The wearable device of embodiment 40, wherein the first ring is proximal to the center and the second ring is distal to the center.

Embodiment 42. The wearable device of embodiments 39 or 40, wherein the one or more biometric sensors is configured on the inner circumferential surface of the first ring.

Embodiment 43. The wearable device of any one of embodiments 1-42, embodied as a ring configured to be placed on and surround a user's finger.

Embodiment 44. A system comprising:
a. one or more wearable devices of any one of embodiments 1-43; and
b. one or more receiver devices configured to communicate with the one or more wearable devices.

Embodiment 45. The system of embodiment 44, wherein the one or more receiver devices comprises:
a. a receiver wireless communication unit;
b. one or more receiver processors;
c. one or more receiver power storage devices;
d. an electromagnet or relay; and
e. receiver memory.

Embodiment 46. The system of embodiment 45, wherein the receiver wireless communication unit comprises a receiver electromagnetic radiation sensor configured to detect the one or more electromagnetic radiation signals emitted from the electromagnetic radiation source of the one or more wearable devices.

Embodiment 47. The system of embodiments 45 or 46, wherein the electromagnet is selected from the group consisting of a solenoid, a servomotor, a stepper motor, and a motor.

Embodiment 48. The system of any one of embodiments 45-47, wherein the one or more receiver devices further comprises a receiver electromagnetic radiation source configured to generate one or more receiver electromagnetic radiation signals, and the one or more wearable devices further comprises an electromagnetic radiation sensor configured to detect the one or more receiver electromagnetic radiation signals emitted from the receiver electromagnetic radiation source of the one or more receiver devices.

Embodiment 49. The system of any one of embodiments 45-48, wherein the receiver memory stores receiver instructions executable by the one or more receiver processors, which when executed cause the one or more receiver devices to:
a. detect a position of the electromagnet;
b. actuate the electromagnet; and
c. reconfigure the position.

Embodiment 50. A system comprising:
a. one or more wearable devices of any one of embodiments 1-43; and
b. one or more smart receiver devices configured to communicate with the one or more wearable devices.

Embodiment 51. The system of embodiment 50, wherein the one or more smart receiver devices comprises:
a. a smart receiver wireless communication unit;
b. one or more smart receiver power storage devices;
c. one or more smart receiver processors; and
d. smart receiver memory.

Embodiment 52. The system of embodiment 51, wherein the smart receiver wireless communication unit comprises a smart receiver electromagnetic radiation sensor configured to detect the one or more electromagnetic radiation signals emitted from the electromagnetic radiation source of the one or more wearable devices.

Embodiment 53. The system of any one of embodiments 50-52, wherein the one or more smart receiver devices further comprises a smart receiver electromagnetic radiation source configured to generate one or more smart receiver electromagnetic radiation signals, and the one or more wearable devices further comprises an electromagnetic radiation sensor configured to detect the one or more smart receiver electromagnetic radiation signals emitted from the smart receiver electromagnetic radiation source of the one or more smart receiver devices.

Embodiment 54. The system of any one of embodiments 51-53, wherein the memory stores instructions executable by the one or more processors, which when executed cause the one or more wearable devices to:
a. detect a smart receiver identification from the smart receiver memory;
b. interface with the internet (directly or indirectly); and
c. cause one or more smart receiver devices to perform one or more functions.

Embodiment 55. The system of any one of embodiments 51-53, wherein the memory stores instructions executable by the one or more processors, which when executed cause the one or more wearable devices to:
a. receive a smart receiver identification transmitted wirelessly by the smart receiver wireless communication unit;
b. interface with the internet (directly or indirectly); and
c. cause one or more smart receiver devices to perform one or more functions.

Embodiment 56. A method of controlling one or more receiver devices, comprising: powering on one or more wearable devices of any one of embodiments 1-43; and activating the one or more input devices.

Embodiment 57. The method of embodiment 56, wherein the activating step comprises touching the one or more wearable devices, producing one or more sound signals, or moving the one or more wearable devices.

Embodiment 58. The method of embodiments 56 or 57, wherein the one or more receiver devices comprises:
a. a receiver wireless communication unit;
b. one or more receiver processors;
c. one or more receiver power storage devices;
d. an electromagnet or relay;
e. one or more position sensors; and
f. receiver memory.

Embodiment 59. The method of embodiment 58, wherein the receiver wireless communication unit comprises a receiver electromagnetic radiation sensor configured to detect the one or more electromagnetic radiation signals emitted from the electromagnetic radiation source of the one or more wearable devices.

Embodiment 60. The method of embodiments 58 or 59, wherein the electromagnet is selected from the group consisting of a solenoid, a servomotor, a stepper motor, and a motor.

Embodiment 61. The method of any one of embodiments 58-60, wherein the receiver memory stores receiver instructions executable by the one or more receiver processors, which when executed cause the one or more receiver devices to:
a. detect a position of the electromagnet;
b. actuate the electromagnet; and
c. reconfigure the position.

Embodiment 62. The method of embodiment 61, further comprising:
a. executing the instructions;
b. detecting the position of the electromagnet;
c. actuating the electromagnet; and
d. reconfiguring the position.

Embodiment 63. The method of any one of embodiments 58-62, wherein the one or more receiver devices further comprises a receiver electromagnetic radiation source configured to generate one or more receiver electromagnetic radiation signals, and the one or more wearable devices further comprises an electromagnetic radiation sensor configured to detect the one or more receiver electromagnetic radiation signals emitted from the receiver electromagnetic radiation source of the one or more receiver devices.

Embodiment 64. The method of embodiment 63, further comprising:
a. receiving the one or more receiver electromagnetic radiation signals emitted by the receiver electromagnetic radiation source; and
b. activating the one or more output devices in response to the one or more receiver electromagnetic radiation signals by wireless communication.

Embodiment 65. A method of controlling one or more smart receiver devices, comprising: powering on one or more wearable devices of any one of embodiments 1-43; and activating the one or more input devices.

Embodiment 66. The method of embodiment 65, wherein the one or more smart receiver devices is configured to communicate with the one or more wearable devices.

Embodiment 67. The method of embodiments 65 or 66, wherein the one or more smart receiver devices comprises:
a. a smart receiver wireless communication unit;
b. one or more smart receiver power storage devices;
c. one or more smart receiver processors; and
d. smart receiver memory.

Embodiment 68. The method of embodiment 67, wherein the smart receiver wireless communication unit comprises a smart receiver electromagnetic radiation sensor configured to detect the one or more electromagnetic radiation signals emitted from the electromagnetic radiation source of the one or more wearable devices.

Embodiment 69. The method of embodiments 67 or 68, wherein the memory stores instructions executable by the one or more processors, which when executed cause the wearable device to:
a. detect a smart receiver identification from the smart receiver memory;
b. interface with the internet (directly or indirectly); and
c. cause the one or more smart devices to perform one or more functions.

Embodiment 70. The method of embodiment 69, further comprising:
a. detecting the smart receiver identification from the smart receiver memory;
b. interfacing with the internet (directly or indirectly); and
c. causing the one or more smart devices to perform one or more functions.

Embodiment 71. A method of controlling one or more receiver devices, comprising:
a. powering on one or more wearable devices of the system of any one of embodiments 44-49; and
b. activating the one or more input devices.

Embodiment 72. The method of embodiment 71, further comprising receiving the one or more electromagnetic radiation signals emitted by the receiver electromagnetic radiation source.

Embodiment 73. The method of embodiment 72, further comprising activating the one or more output devices in response to the one or more receiver electromagnetic radiation signals.

Embodiment 74. A method of controlling one or more smart receiver devices, comprising:
a. powering on one or more wearable devices of the system of any one of embodiments 50-55; and
b. activating the one or more input devices.

Embodiment 75. The method of embodiment 74, further comprising:
a. receiving a smart receiver identification transmitted wirelessly by the smart receiver wireless communication unit;
b. interfacing with the internet (directly or indirectly); and
c. causing the one or more smart receiver devices to perform the one or more functions.

1-75. (canceled)

76. A wearable device comprising:
a ring-shaped body;
one or more input devices provided on at least one region of an inner circumferential surface or on at least one region of an outer circumferential surface of the ring-shaped body, a user configured to activate the one or more input devices by touching the one or more input devices; and
one or more output devices, the one or more output devices configured to send an electromagnetic radiation signal in response to the user activating the one or more input devices.

77. The wearable device of claim 76, wherein the one or more input devices includes at least one of a touch sensor or a button.

78. The wearable device of claim 76, further comprising one or more power storage devices configured to store electric power, wherein the one or more power storage devices is removable and interchangeable.

79. The wearable device of claim 78, wherein the one or more power storage devices is configured for inductive or non-inductive wireless charging by pressure contact, and wherein magnets are used for pressure contact.

80. The wearable device of claim 78, further comprising one or more energy harvesting devices, wherein the one or more energy harvesting devices are configured to transmit electric power to the one or more power storage devices, and wherein the one or more energy harvesting devices includes at least one of a thermoelectric generator or a transducer.

81. The wearable device of claim 76, wherein the one or more output devices includes at least one electromagnetic radiation source configured to generate the electromagnetic radiation signal, wherein the electromagnetic radiation signal includes at least one of a visible light signal or an invisible light signal.

82. The wearable device of claim 81, wherein the electromagnetic radiation signal includes the invisible light signal, the invisible light signal includes at least one of infrared radiation or radio frequency radiation.

83. The wearable device of claim 76, further comprising:
one or more wireless communication units configured for wireless communication with one or more hubs over at least one of a Bluetooth protocol or a mesh protocol.

84. The wearable device of claim 83, wherein the one or more hubs includes at least one of a charging station, a mobile device, or a smart device.

85. The wearable device of claim 76, further comprising a tactile surface configured to orient the wearable device with respect to a user's finger.

86. The wearable device of claim 76, wherein the ring-shaped body is configured to be placed on and surround a user's finger.

87. A system, comprising:
a wearable device comprising:
a ring-shaped body;
one or more input devices provided on at least one region of an inner circumferential surface or on at least one region of an outer circumferential surface of the ring-shaped body; and
one or more output devices provided on at least one region of the inner circumferential surface or at least one region of the outer circumferential surface; and
one or more receiver devices configured to receive a signal from the one or more output devices.

88. The system of claim 87, wherein the one or more receiver devices includes a receiver electromagnetic radiation sensor configured to detect one or more electromagnetic radiation signals emitted from the one or more output devices of the wearable device.

89. The system of claim 87, wherein the one or more receiver devices includes an electromagnet, wherein the electromagnet includes at least one of a solenoid, a servomotor, a stepper motor, or a motor.

90. The system of claim 89, wherein the one or more receiver devices includes a memory that stores instructions executable by one or more processors of the one or more receiver devices, which when executed cause the one or more receiver devices to:
detect a position of the electromagnet;
actuate the electromagnet; and
reconfigure the position of the electromagnet.

91. The system of claim 89, wherein the one or more receiver devices is configured to be installed over a toggle light switch such that the toggle light switch can be (1) operated in response to the signal from the one or more output devices and (2) manually operated by a user.

92. The system of claim 89, wherein the one or more receiver devices includes magnets configured to magnetically couple the one or more receiver devices to a toggle light switch.

93. A method, comprising:
receiving an input signal in response to a user activating an input device on a surface of a ring-shaped device; and
sending, from an output device of the ring-shaped device and in response to the input signal, one or more electromagnetic radiation signals such that a receiver device receives the one or more electromagnetic radiation signals and activates a function of the receiver device in response to the one or more electromagnetic radiation signals.

94. The method of claim 93, wherein the activating the input device includes at least one of the user touching the ring-shaped device or the user moving the ring-shaped device.

95. The method of claim 93, wherein the receiver device includes a receiver electromagnetic radiation sensor configured to detect the one or more electromagnetic radiation signals emitted from the output device.

96. The method of claim 93, wherein the receiver device includes an electromagnet including at least one of a solenoid, a servomotor, a stepper motor, or a motor.

97. The method of claim 96, further comprising:
detecting a position of the electromagnet;
actuating the electromagnet; and
reconfiguring the position of the electromagnet.
